(19) United States
(12) Patent Application Publication
(10) Pub. No.: US 2018/0016289 A1
(43) Pub. Date: Jan. 18, 2018

(54) REDOX-SENSITIVE VESICLES

(71) Applicants: THE ROYAL INSTITUTION FOR THE ADVANCEMENT OF LEARNING/MCGILL UNIVERSITY, MONTREAL (CA); TRANSFERT PLUS SOCIÉTÉ EN COMMANDITE, MONTREAL (CA)

(72) Inventors: JANINE MAUZEROLL, MONTREAL (CA); TOMER AHARON MOYHOUZER, MONTREAL (CA); MICHAEL EDWARD SNOWDEN, MONTREAL (CA); PHILIPPE DAUPHIN DUCHARME, MONTREAL (CA); STEPHANI MAZURKIEWICZ, MONTREAL (CA); CHLOÉ L'HOMME, LA HAYE DU PUITS (FR); SAMUEL DESJARDINS, LAVAL (CA); SYLVAIN CANESI, MONTREAL (CA)

(21) Appl. No.: 15/541,559

(22) PCT Filed: Jan. 19, 2016
(86) PCT No.: PCT/CA2016/050039

§ 371 Date: Jul. 5, 2017

Related U.S. Application Data
(60) Provisional application No. 62/105,039, filed on Jan. 19, 2015.

(51) Int. Cl. C07F 17/02; A61K 9/127; A61K 31/704; A61K 47/24

(52) U.S. Cl. CPC C07F 17/02; A61K 47/24; A61K 9/127; A61K 31/704

(57) ABSTRACT

There is provided a redox-sensitive compound comprising a redox-sensitive organometallic moiety and a phospholipid or modified-phospholipid moiety. The compound is embodied in a redox-sensitive drug delivery system. In preferred embodiments, the system comprises redox active giant unilamellar vesicles (GUVs), which are used as drug delivery vessels.

### FIELD OF THE INVENTION

The invention relates generally to the controlled-release of biologically active agents to body sites of humans and animals. More specifically, the invention relates to a redox-sensitive drug delivery system.

### BACKGROUND OF THE INVENTION

The field of drug delivery systems is an emerging field still presenting many challenges. There is a wide interest in developing an efficient and reliable system that is able to transport a biologically active material to a desired location, and then releases it through a simple process [1-4]. Various approaches have been explored with the aim of mitigating problems that arise with the use of such system. Typically, these problems are nonspecific distribution in the body, poor solubility, diffusion inside the transport vessel or in the body, drug release profile and nonspecific trigger mechanism.

Liposomes are the most common drug delivery systems used today. Indeed, they can be non-toxic, biodegradable and biocompatible [5]. Furthermore, their nature enables them to be tailored made in terms of size, nature (hydrophobic or hydrophilic shell) and functionality [6,7]. Another advantage associated with the use of liposomes is the ability to incorporate different substances within the inner void during or after (remote loading) the assembly process [6]. The trigger mechanism may be a physical property such as pH [8,9] or temperature [10]. Also, the trigger mechanism may utilize a specific recognition property such as an antibody [11,12], an enzyme [13,14] or a ligand [15]. Other means of release mechanism include the use of oscillation waves (ultrasound) [16] and photochemistry [17,18].

The inventors are also aware of the following patent documents: U.S. 2011-0104250, EP 1225873, U.S. Pat. No. 6,726,925, U.S. 2011-0275980, U.S. 2012-0129270 [19].

There is still a need for drug delivery systems. In particular, there is a need for drug delivery systems that are easy to prepare, cost-efficient and that have a simple and reliable trigger mechanism.

The present description refers to a number of documents, the content of which is herein incorporated by reference in their entirety.

### SUMMARY OF THE INVENTION

The inventors have designed and prepared a drug delivery system that is redox-sensitive. The system comprises a redox-sensitive compound, which is a redox-sensitive phospholipid or modified-phospholipid. The redox-sensitive compound according to the invention comprises a redox-sensitive moiety and a phospholipid or modified-phospholipid moiety. In embodiments of the invention, the two moieties are attached together by a linker, which is a C₁ to C₈ alkyl group optionally comprising at least one of C═O, C═S, C═N and O═S═O, optionally the backbone of the linker comprises at least one heteroatom selected from O, S and N.

The invention thus provides for the following according to aspects thereof:
(1) A redox-sensitive compound comprising a redox-sensitive organometallic moiety and a phospholipid or modified-phospholipid moiety, optionally the two moieties are attached together by a linker which is a C₁ to C₈ alkyl group optionally comprising at least one of C═O, C═S, C═N and O═S═O, optionally the backbone of the linker comprises at least one heteroatom selected from O, S and N.
(2) A redox-sensitive compound having a general formula 3A

Q-L-U 3A
wherein:
   Q is a redox-sensitive organometallic group, preferably the metal is selected from Fe, Ir, Ru and Pt;
   L, which is present or absent, is a linker which is a C₁ to C₈ alkyl group optionally comprising at least one of C═O, C═S, C═N and O═S═O, optionally the backbone of the linker comprises at least one heteroatom selected from O, S and N; and
   U is a phospholipid or modified-phospholipid moiety.
(3) A redox-sensitive compound having a general formula 3B

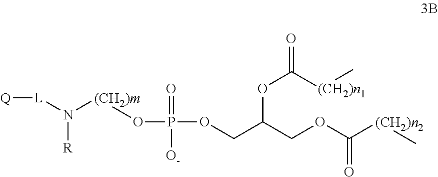

wherein:
   Q is a redox-sensitive group, preferably the group comprises at least one metal atom, preferably the metal atom is selected from Fe, Ir, Ru and Pt;
   X which is present or absent, is a linker which is a C₁ to C₈ alkyl group optionally comprising at least one of C═O, C═S, C═N and O═S═O, optionally the backbone of the linker comprises at least one heteroatom selected from O, S and N;
   R is a C₁ to C₈ alkyl group;
   m is an integer selected from 1 to 8; and
   n₁ and n₂ are each independently an integer selected from 1 to 30.
(4) A redox-sensitive compound having a general formula 3C

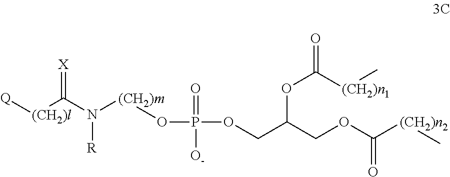

wherein:
Q is a redox-sensitive group, preferably the group comprises at least one metal atom, preferably the metal atom is selected from Fe, Ir, Ru and Pt;
X is a heteroatom selected from O, S and N;
R is a C₁ to C₈ alkyl group;
l and m are each independently an integer selected from 1 to 8; and
n₁ and n₂ are each independently an integer selected from 1 to 30.
(5) A redox-sensitive compound having a general formula 3D

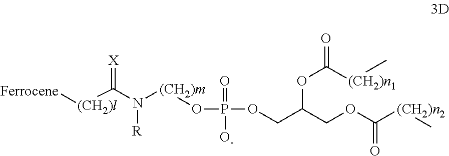

Wherein:
   X is a hetero atom selected from O, S and N;
   R is a C₁ to C₈ alkyl group;
   l and m are each independently an integer selected from 1 to 8; and
   n₁ and n₂ are each independently an integer selected from 1 to 30.
(6) A redox-sensitive compound having a general formula 3E

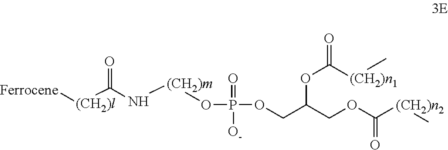

wherein:
   l and m are each independently an integer selected from 1 to 8; and
   n₁ and n₂ are each independently an integer selected from 1 to 30.
(7) A redox-sensitive phospholipid having a general formula 3F

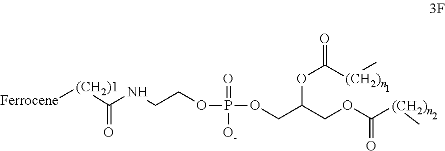

wherein:
   l is an integer selected from 1 to 8; and
   n₁ and n₂ are each independently an integer selected from 1 to 30.
(8) A redox-sensitive phospholipid of formula 3

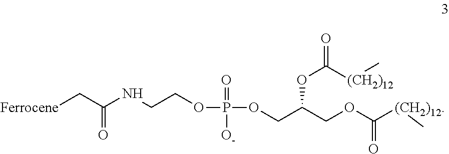

(9) A redox-sensitive drug delivery system comprising a redox-sensitive compound as defined in any one of (1) to (8).
(10) A redox-sensitive drug delivery system comprising a redox-sensitive compound as defined in any one of (1) to (8), and at least one phospholipid compound that is not redox-sensitive.
(11) A redox-sensitive drug delivery system comprising a redox-sensitive compound as defined in any one of (1) to (8), and two phospholipid compounds that are not redox-sensitive.
(12) A redox-sensitive drug delivery system comprising a redox-sensitive phospholipid of formula 3E as defined in (6) or formula 3F as defined in (7).
(13) A redox-sensitive drug delivery system comprising a redox-sensitive phospholipid of formula 3E as defined in (6) or formula 3F as defined in (7), and at least one phospholipid compound that is not redox-sensitive.
(14) A redox-sensitive drug delivery system comprising a redox-sensitive phospholipid of formula 3E as defined in (6) or formula 3F as defined in (7), and first and second phospholipid compounds that are not redox-sensitive.
(15) A redox-sensitive drug delivery system according to (14), wherein, when the redox-sensitive phospholipid is of formula 3E, the first phospholipid compound that is not redox-sensitive is a compound of general formula 2A outlined below and the second phospholipid that is not redox-sensitive is a compound of general formula 4A outlined below; and when the redox-sensitive phospholipid is of formula 3F, the first phospholipid compound that is not redox-sensitive is a compound of general formula 2A′ outlined below

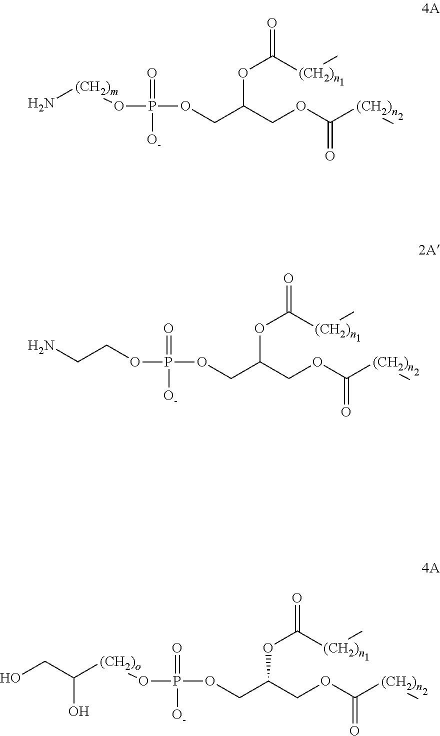

wherein:
   m and o and each independently an integer selected from 1 to 8; and
   n₁ and n₂ are each independently an integer selected from 1 to 30.
(16) A redox-sensitive drug delivery system comprising the redox-sensitive phospholipid of formula 3 as defined in (8).
(17) A redox-sensitive drug delivery system comprising the redox-sensitive phospholipid of formula 3 as defined in (8), and at least one phospholipid that is not redox-sensitive.
(18) A redox-sensitive drug delivery system comprising the redox-sensitive phospholipid of formula 3 as defined in (8), and first and second phospholipid compounds that are not redox-sensitive.
(19) A redox-sensitive drug delivery system according to (18), wherein the first phospholipid compound that is not redox-sensitive is a compound of general formula 2 outlined below and the second phospholipid that is not redox-sensitive is a compound of general formula 4 outlined below

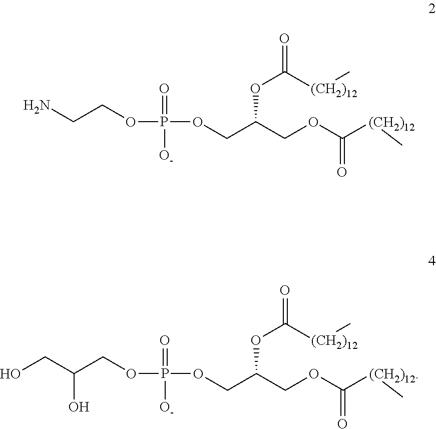

(20) A redox-sensitive drug delivery system according to any one of (9) to (19), wherein at least part of the redox-sensitive groups of the redox-sensitive phospholipid is located on an outer surface of the system.
(21) A redox-sensitive drug delivery system according to any one of (12) to (15), wherein at least part of the ferrocene groups of the redox-sensitive phospholipid 3E or 3F is located on an outer surface of the system.
(22) A redox-sensitive drug delivery system according to any one of (16) to (19), wherein at least part of the ferrocene groups of the redox-sensitive phospholipid 3 is located on an outer surface of the system.
(23) A redox-sensitive drug delivery system according to (11), wherein the redox-sensitive phospholipid and one of the two phospholipid compounds are present in a molar ratio phospholipid compound:redox-sensitive phospholipid between about 1:0.01 to about 1:1, preferably between about 1:0.1 to about 1:0.8, more preferably between about 1:0.2 to about 1:0.6.
(24) A redox-sensitive drug delivery system according to (15), wherein the redox-sensitive phospholipid 3E and the first phospholipid compound 2A or the redox-sensitive phospholipid 3F and the first phospholipid compound 2A′ are present in a molar ratio 2A:3E or 2A′:3F between about 1:0.01 to about 1:1, preferably between about 1:0.1 to about 1:0.8, more preferably between about 1:0.2 to about 1:0.6.
(25) A redox-sensitive drug delivery system according to (17), wherein the redox-sensitive phospholipid 3 and the first phospholipid compound 2 are present in a molar ratio 2:3 between about 1:0.01 to about 1:1, preferably between about 1:0.1 to about 1:0.8, more preferably between about 1:0.2 to about 1:0.6.
(26) A redox-sensitive drug delivery system according to any one of (9) to (25), having a size between about 100 nm to 40 μm, preferably between about 100 nm to 700 nm, more preferably between about 200 nm to 500 nm.
(27) A method for preparing a redox-sensitive phospholipid of formula 3E, comprising reacting a redox-sensitive compound of formula 1A and a phospholipid of formula 2A as outlined below

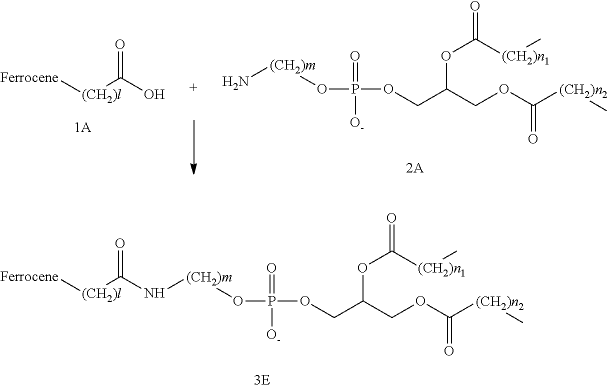

wherein:
   l and m and each independently an integer selected from 1 to 8; and
   n₁ and n₂ are each independently an integer selected from 1 to 30.
(28) A method for preparing a redox-sensitive drug delivery system, comprising (a) providing a redox-sensitive phospholipid of formula 3E; and (b) mixing the redox-sensitive phospholipid of formula 3E and a first phospholipid of formula 2A in the presence of a second phospholipid of formula 4A outlined below

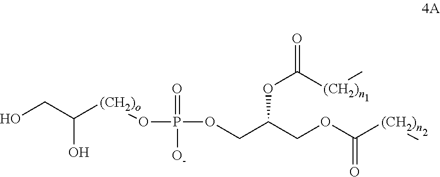

wherein:
   o is an integer selected from 1 to 8; and
   n₁ and n₂ are each independently an integer selected from 1 to 30.
(29) A method for preparing a redox-sensitive drug delivery system, comprising the following steps:
   (a) reacting a redox-sensitive compound of formula 1A and a first phospholipid of formula 2A to obtain a redox-sensitive phospholipid of formula 3E; and
   (b) mixing the redox-sensitive phospholipid of formula 3E and the first phospholipid of formula 2A in the presence of a second phospholipid of formula 4A to obtain the redox-sensitive drug delivery system.
(30) A method according to (28) or (29), wherein the second phospholipid of formula 4A is present in catalytic amount.
(31) A method for preparing a redox-sensitive phospholipid of formula 3, comprising reacting a redox-sensitive compound of formula 1 and a phospholipid of formula 2 as outlined below

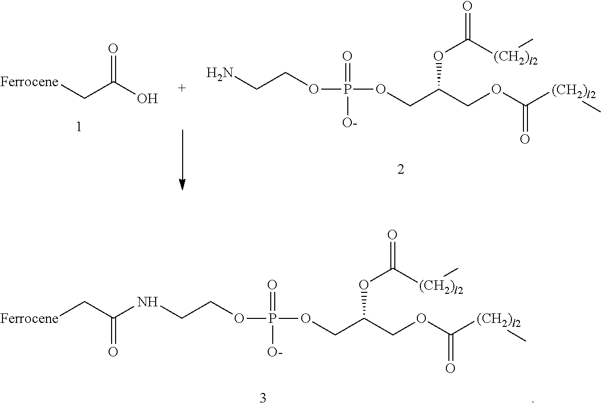

(32) A method for preparing a redox-sensitive drug delivery system, comprising: (a) providing a redox-sensitive phospholipid of formula 3; and (b) mixing the redox-sensitive phospholipid of formula 3 and a first phospholipid of formula 2 in the presence of a second phospholipid of formula 4 outlined below

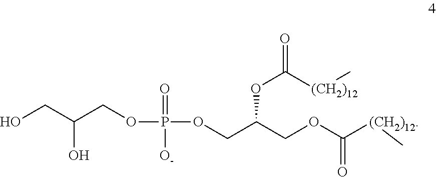

(33) A method for preparing a redox-sensitive drug delivery system, comprising the following steps:
   (a) reacting a redox-sensitive compound of formula 1 and a first phospholipid compound of formula 2 to obtain a redox-sensitive phospholipid of formula 3; and
   (b) mixing the redox-sensitive phospholipid of formula 3 and the first phospholipid of formula 2 in the presence of a second phospholipid of formula 4 to obtain the redox-sensitive drug delivery system.
(34) A method according to (32) or (33), wherein the second phospholipid of formula 4 is present in catalytic amount.
(35) A method according to (28) or (29), wherein, during step (b) the redox-sensitive phospholipid 3E and the first phospholipid compound 2A self-assemble to form the system, and at least part of the ferrocene groups are located on an outer layer of the system.
(36) A method according to (32) or (33), wherein, during step (b) the redox-sensitive phospholipid 3 and the first phospholipid compound 2 self-assemble to form the system, and at least part of the ferrocene groups are located on an outer layer of the system.
(37) A method as defined in any one of (28), (29), (33) and (34), further comprising a step (c) of subjecting the redox-sensitive drug delivery system to filtration to obtain batches of redox-sensitive drug delivery system wherein vesicles in a batch have a given average size range.
(38) A method according to (37), wherein at least two separate batches are obtained as follows: a batch of vesicles with a range of average diameters of about 80-300 nm, and a batch of larger vesicles with a range of average diameters of about 500 nm to 1 μm.
(39) A method according to (37) or (38), wherein the redox-sensitive drug delivery system is isolated and stored.
(40) A method according to (39), wherein the storage is at room temperature or in a refrigeration or freezing system.
(41) A redox-sensitive drug delivery system which is obtained by a method as defined in any one of (27) to (40).
(42) A method for preparing a loaded redox-sensitive drug delivery system, comprising: (a) providing a redox-sensitive drug delivery system as defined in any one of (9) to (25); and (b) mixing the redox-sensitive drug delivery system and a biologically active agent.
(43) A method for preparing a loaded redox-sensitive drug delivery system, comprising: (a) providing a redox-sensitive phospholipid of formula 3E; and (b) mixing the redox-sensitive phospholipid of formula 3E, a first phospholipid of formula 2A, a second phospholipid of formula 4A, and a biologically active agent.
(44) A method for preparing a loaded redox-sensitive drug delivery system, comprising: (a) providing a redox-sensitive phospholipid of formula 3; and (b) mixing the redox-sensitive phospholipid of formula 3, a first phospholipid of formula 2, a second phospholipid of formula 4, and a biologically active agent.
(45) A method according to any one of (42) to (44), wherein the biologically active agent is encapsulated within the system.
(46) A method according to any one of (42) to (44), wherein, during step (b) the redox-sensitive phospholipid and the first phospholipid self-assemble to form the system and the biologically active agent is encapsulated within the system, in situ.
(47) A method according to any one of (42) to (46), wherein the biologically active agent is selected from: antitumor agents, antibiotics, anthracycline antibiotics, immunodilators, anti-inflammatory drugs, drugs acting on the central nervous system, proteins, peptides, doxorubicin, daunorubicin, epirubicin, idarubicin, and mitoxantrone.
(48) A method according to any one of (42) to (46), wherein the biologically active agent is a chemotherapeutic agent.
(49) A loaded redox-sensitive drug delivery system which is obtained by the method as defined in any one of (42) to (48).
(50) A loaded redox-sensitive drug delivery system according to (49), which is specific to cancer cells.
(51) A pharmaceutical composition comprising a loaded redox-sensitive drug delivery system as defined in (49), and a pharmaceutically acceptable carrier.
(52) A method of treating a medical condition in a human or animal, comprising administering to the human or animal a loaded redox-sensitive drug delivery system as defined in (49) or a pharmaceutical composition as defined in (51), and wherein the loaded biologically active agent is for treating the medical condition.
(53) Use of a loaded redox-sensitive drug delivery system as defined in (49) or a pharmaceutical composition as defined in (48), for treating a medical condition in a human or animal, wherein the loaded biologically active agent is for treating the medical condition.
(54) Use of a loaded redox-sensitive drug delivery system as defined in (49), in the manufacture of a medicament for treating a medical condition in a human or animal, wherein the loaded biologically active agent is for treating the medical condition.
(55) A loaded redox-sensitive drug delivery system as defined in (49) or a pharmaceutical composition as defined in (51), for use in the treatment of a medical condition in a human or animal, wherein the loaded biologically active agent is for treating the medical condition.
(56) A method according to (52) or a use according to (53) or (54), wherein the biologically active agent is released upon contact of the loaded system with an oxidant; preferably the oxidant is Ir^{(IV)}Cl₆²⁻.
(57) A method according to (52) or a use according to (53), (54) or (56), wherein the biologically active agent is released upon contact of the loaded system with a biological system; preferably the biological system comprises cancer cells; more preferably the biological system comprises HeLa cells.
(58) A method according to (52) or a use according to (53), (54) or (56), wherein the biologically active agent is released upon contact of the loaded system with cancer cells and the biologically active agent is not released upon contact of the loaded system with other cells.
(59) A research platform, which embodies a redox-sensitive compound as defined in any one of (1) to (8).
(60) A research platform, which embodies a redox-sensitive compound as defined in any one of (1) to (8) and at least one phospholipid compound that is not redox-sensitive.
(61) A research platform, which embodies a redox-sensitive phospholipid of formula 3E, a first phospholipid of formula 2A and a second phospholipid of formula 4A.
(62) A research platform, which embodies a redox-sensitive phospholipid of formula 3F, a first phospholipid of formula 2A′ and a second phospholipid of formula 4A.
(63) A research platform, which embodies a redox-sensitive phospholipid system of formula 3, a first phospholipid of formula 2 and a second phospholipid of formula 4.
(64) A research platform, which embodies a redox-sensitive drug delivery system as defined in any one of (9) to (26).
(65) Use of a research platform as defined in any one of (59) to (64), in drug discovery or drug screening.

Other objects, advantages and features of the present invention will become more apparent upon reading of the following non-restrictive description of specific embodiments thereof, given by way of example only, with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In the appended drawings:

FIG. 1: An embodiment of the invention including the preparation of a redox-sensitive phospholipid compound and a redox-sensitive delivery system according to the invention. The payload release is also illustrated. Moreover, a graphical representation of the phospholipid and the system is illustrated.

FIG. 2: Preparation of a redox-sensitive phospholipid compound according to the invention.

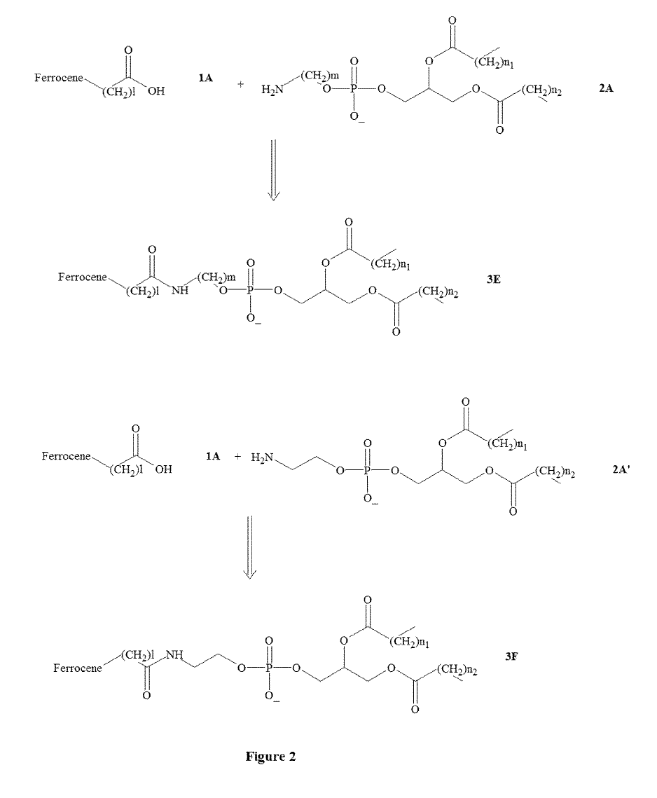

FIG. 3: Preparation of a redox-sensitive delivery system according to the invention.

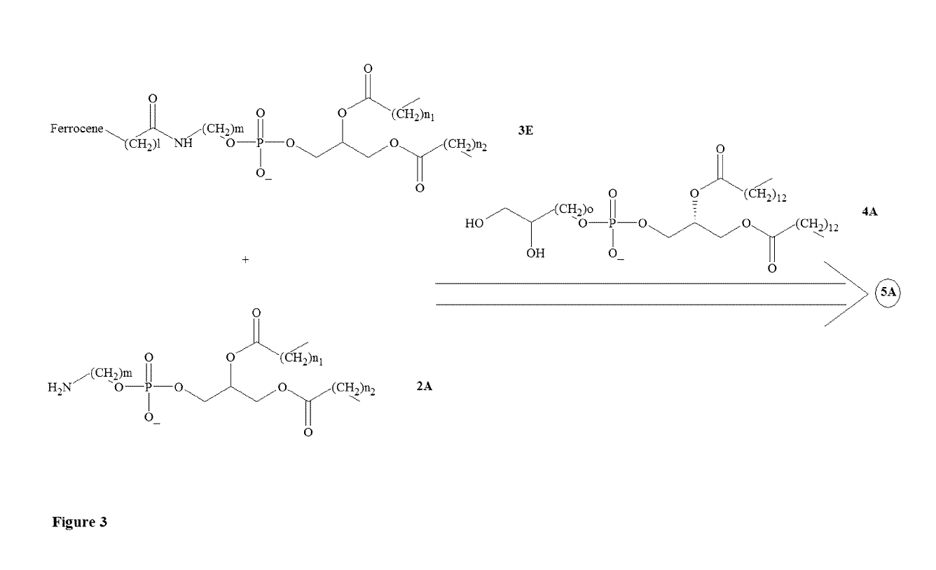

FIG. 4: Preparation of a redox-sensitive phospholipid compound according to the invention.

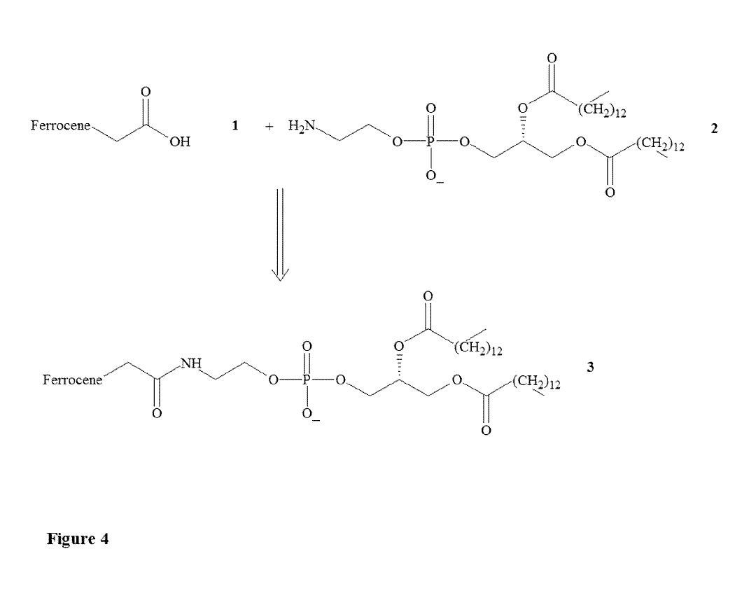

FIG. 5: Preparation of a redox-sensitive delivery system according to the invention.

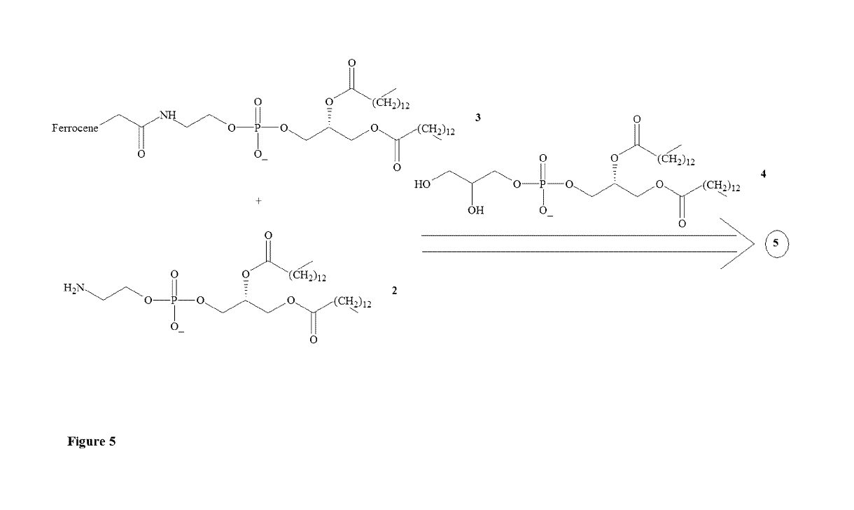

FIG. 6: Cyclic voltammetry (CV) measurements conducted in acetonitrile using 0.1 M of TBA-PF₆ as the electrolyte. All the measurements were conducted at a scan rate of 50 mV.s⁻¹ using a glassy carbon (Ø=3 mm) electrode as a working electrode, Pt wire as a counter electrode and Ag wire coated with AgCl as a reference electrode. (A) Voltammogram of a 1 mM ferroceneacetic acid solution (B) voltammogram of a 1 mM phospholipid 2 solution. (C) Voltammogram of a 1 mM phospholipid 3 solution.

FIG. 7: (A) shows a micrograph of redox-active GUVs, (B) a fluorescent micrograph of redox-active GUVs were loaded with calcein and (C) an overlay of fluorescent and transmitted light (TL) micrographs of GUVs loaded with calcein (B).

FIG. 8: An overlay of a fluorescent and transmitted light micrographs of a redox-active GUV before (A) and after (B), 5 minutes by illumination.

FIG. 9: (A) CV of the Pt-UME in the bulk solution that contains 1 mM K₃Ir^{(III)}Cl₆ in 0.1 M KCl and 50 mM Glucose. (B) SECM approach curves: Glass substrate theoretical (black-dot) and experimental (Black), phospholipid 2: phospholipid 3 ratio of 1:0.2 theoretical (blue-dot) and experimental (blue), phospholipid 2: phospholipid 3 ratio of 1:0.4 theoretical (red-dot) and experimental (red).

FIG. 10: Micrographs of redox active and regular GUVs that were tagged with FC-Ab1 and fluorescent Ab2. Redox active: (A) transmitted light (B) fluorescent and (C) an overly of (A) and (B). Regular GUVs (D) transmitted light (E) fluorescent and (F) an overly of (D) and (E).

FIG. 11: A transferred light micrograph of a 1:0.4 ratio GUV before (A) and after an addition of K₂Ir^{(IV)}Cl₆. (B) 7 ms (C) 34 ms (D) 45 ms (E) 52 ms (F) 71 ms. The payload release is clearly visible.

FIG. 12: DLS measurement of filtered 1:0.4 ratio LUV population before (black) and after (red) an addition of K₂Ir^{(IV)}Cl₆.

FIG. 13: Fluorescent intensity measurements sucrose solution before and after addition of regular LUVs (A) or redox-active LUVs (B) and then adding K₂Ir^{(IV)}Cl₆ (C) the same as (A) and (B) only without the addition of the LUVs.

FIG. 14: (A) Synthesis of Fc-DSP (3) from ferroceneacetic acid (1) and DSPE (2) using a detailed reaction and another graphical representation that are used in FIGS. 15-17 (B) CV measurements conducted in acetonitrile using 0.1 M of TBA-PF₆ as the electrolyte. All measurements were conducted at a scan rate of 50 mV.s⁻¹ using a glassy carbon (Ø=3 mm) electrode as a working electrode, Pt wire as a counter electrode and Ag wire coated with AgCl as a reference electrode. (black) Voltammogram of a 1 mM phospholipid 2 solution. (red) Voltammogram of 1 mM phospholipid 3 solution.

FIG. 15: (left) The self-assembly formation of unilamellar giant vesicles (redox active (6) and non-redox-active (7)) from a mixture of (2), DSPC (4) and DSPG (5) followed by the fluorescent antibody labelling using Fc-Ab1 and Ab2. (right) the fluorescent micrographs of the labelled vesicles.

FIG. 16: (top) Redox active GUVs that showed a payload release upon addition of Ir^{(IV)}Cl₆²⁻ and were examined using SECM (see FIG. 20) and transmitted light microscopy (see FIG. 23). (bottom) Redox active LUVs that showed a payload release upon addition of Ir^{(IV)}Cl₆²⁻ and were examined using a pH sensitive fluorescent) and DLS (see FIG. 24) measurements.

FIG. 17: In vitro experiments using GUVs loaded with doxorubicin. (A) Fluorescent microscopy images of HeLa cells that were exposed to redox and non-redox active GUVs. A significant amount of doxorubicin is only observed in the HeLa exposed to the redox active GUVs. (B) Flow cytometry of HeLa and MRC-5 cells exposed to the control (black), redox (red) and non-redox (green) active GUVs. The only significant effect was observed in the HeLa cells exposed to redox active GUVs.

FIG. 18: Process diagrams involving GUVs Imaging (A) Indirect immunofluorescence imaging (B) Preparing the doxorubicin loaded GUVs for the live cells experiments (flow cytometry or imaging).

FIG. 19: Cyclic voltammetry (CV) measurement of (A) 1 mM ferroceneacetic acid solution in acetonitrile using 0.1 M of TBA-PF₆ as the electrolyte. (B) Three repeated voltammograms (solid, dashed and dotted lines, respectively) of a 1 mM phospholipid 3 solution. All conditions are identical as described in FIG. 14 (C) 0.1 M of TBA-PF₆ in acetonitrile before (black) and after (red) the solution was purge for 30 minutes using N₂. The damping of the oxygen reduction peak is clearly observed.

FIG. 20: SECM approach curves above a glass substrate (black), phospholipid 4:phospholipid 3 ratio of 1:0.2 (blue) and phospholipid 4:phospholipid 3 ratio of 1:0.4 (red). Theoretical (dotted) as well as experimental (full) approach curves are presented. Insert CV of the Pt-UME in the bulk solution that contains 1 mM K₃Ir^{(IV)}Cl₆ in 0.1 M KCl and 50 mM glucose.

FIG. 21: TEM and corresponding EDX results of GUVs (ratio 1:0.4). The results with the lowest and highest iron percentages are presented.

FIG. 22: Micrographs of redox and non-redox active GUVs that were tagged with FC-Ab1 and fluorescent Ab2. Redox active: (A) transmitted light (B) fluorescent overlay and (C) fluorescent channel. Non-redox GUVs (D) transmitted light (E) fluorescent overlay and (F) fluorescent channel.

FIG. 23: A transferred light micrograph of a 1:0.4 ratio GUVs before (A) and after an addition of K₂Ir^{(IV)}Cl₆. (B) 8 ms (C) 34 ms (D) 45 ms (E) 54 ms (F) 91 ms. The payload release is clearly visible. The scale bars for all the images is 50 μm.

FIG. 24: DLS measurement of filtered (A) non-redox active LUV population before (black) and after (red) addition of K₂Ir^{(IV)}Cl₆ (B) 1:0.4 ratio redox active LUV population before (black) and after (red) an addition of K₂Ir^{(IV)}Cl₆.

FIG. 25: Optical micrographs of HeLa cells that were exposed to redox active GUVs for 5 hours (A) fluorescent channel overlapping the bright field micrograph (B) fluorescent channel that corresponds to a (C) fluorescent channel overlapping the dark field micrograph (D) fluorescent channel that corresponds to c.

FIG. 26: Optical micrographs of HeLa cells that were exposed to non-redox active GUVs for 5 hours (A) fluorescent channel overlapping the bright field micrograph (B) fluorescent channel that corresponds to a (C) fluorescent channel overlapping the dark field micrograph (D) fluorescent channel that corresponds to c.

FIG. 27: The difference in fluorescence intensity between (A) HeLa and (B) MRC-5 untreated cells, cells treated with non-redox GUVs and cells treated with redox GUVs as measured by flow cytometry.

### DESCRIPTION OF ILLUSTRATIVE EXAMPLES AND EMBODIMENTS

Before the present invention is further described, it is to be understood that the invention is not limited to the particular embodiments described below, as variations of these embodiments may be made and still fall within the scope of the appended claims. It is also to be understood that the terminology employed is for the purpose of describing particular embodiments, and is not intended to be limiting. Instead, the scope of the present invention will be established by the appended claims.

In order to provide a clear and consistent understanding of the terms used in the present disclosure, a number of definitions are provided below. Moreover, unless defined otherwise, all technical and scientific terms as used herein have the same meaning as commonly understood to one of ordinary skill in the art to which this disclosure pertains.

As used herein, the term “phospholipid” is intended to refer to a compound that comprises the components of a phospholipid, namely, a hydrophobic tail consisting of two hydrocarbon chains and a hydrophilic head consisting of choline, phosphate and glycerol.

As used herein, the term “modified-phospholipid” is intended to refer to a compound that comprises a hydrophobic tail consisting of two hydrocarbon chains and a hydrophilic head which is a modified version of a regular phospholipid hydrophilic head; for example, the ethane part of choline may be a C₁ to C₈ alkyl optionally substituted.

As used herein, the term “redox-sensitive compound” is intended to refer to a compound comprising a moiety that is capable of undergoing electrons transfer including loss of electrons (oxidation) or gain of electrons (reduction).

As used herein, the term “redox-sensitive organometallic group” is intended to refer to a group comprising at least one transition metal atom and that is capable of undergoing electrons transfer including loss of electrons (oxidation) or gain of electrons (reduction).

As used herein, the term “drug delivery system” is intended to refer to a system for transporting a biologically active agent in the body of a human or animal such as to deliver the agent at a targeted site within the body.

As used herein, the term “redox-sensitive drug delivery system” is intended to refer to a delivery system that embodies a redox-sensitive assembly capable of releasing its drug payload through redox chemistry involving electrons transfer including loss of electrons (oxidation) or gain of electrons (reduction).

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “about” is used to indicate that a value includes an inherent variation of error for the device or the method being employed to determine the value.

The inventors have designed and prepared a drug delivery system that is redox-sensitive. The system comprises a redox-sensitive compound, which is a redox-sensitive phospholipid or modified-phospholipid. The redox-sensitive compound according to the invention comprises a redox-sensitive moiety and a phospholipid or modified-phospholipid moiety. In embodiments of the invention, the two moieties are attached together by a linker, which is a C₁ to C₈ alkyl group optionally comprising at least one of C═O, C═S, C═N and O═S═O, optionally the backbone of the linker comprises at least one heteroatom selected from O, S and N.

As will be understood by a skilled person, a biologically active agent may be encapsulated within the redox-sensitive delivery system of the invention, such that a targeted delivery of the agent to a body site of a human or animal may be performed.

Also as will be understood by a skilled person, the redox-sensitive delivery system of the invention may be a useful tool in drug discovery or drug screening.

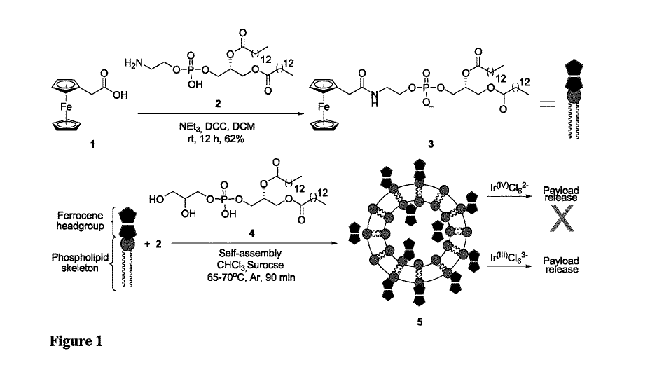

The present disclosure relates to a modified ferrocene phospholipid that may be used to form a redox triggered vesicles as described in FIG. 1. Redox triggering is sensitive to small and local changes; therefore it may be applied without affecting other species in the environment as opposed to pH, temperature, ultrasound and photochemistry changes. Moreover, the triggering mechanism may be designed to be specific such as to avoid unwanted payload release.

Only a few examples are reported in the literature [20-22] on the modification or formation of a phospholipid bearing a ferrocenyl segment. Such modification or formation is produced by organic synthesis transformations. Saji et al. [20] published a paper in 1985 where they demonstrated the synthesis of a ferrocene bearing surfactant. They also hypothesized that this surfactant could be used for a formation of a redox triggered micelle. McCarley and coworkers [23-25] developed a one-step approach and further demonstrated that quinone modified phospholipid could be used in various applications such as drug delivery vectors.

The approach according to the invention is based on a formation of liposome (vesicles) modified with an inorganic ferrocene moiety, as opposed to an organic quinone one. This organometallic complex is a stable compound with a defined outer sphere electron transfer mechanism [26], while quinones in general are less stable and tend to oxidize in natural environments; they also exhibit a much more complex electron transfer process.

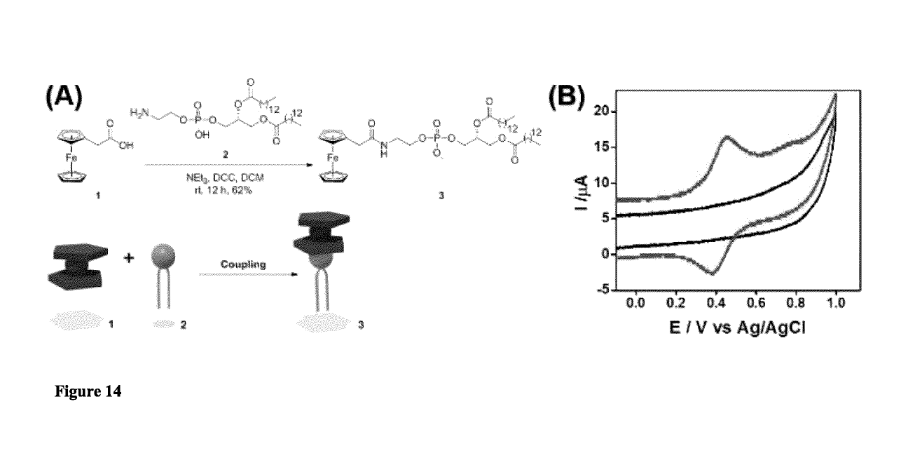

The inventors characterized the ferrocene modified phospholipid (3) using NMR and electrochemistry, and investigated the stability of the redox active giant unilamellar vesicles (GUVs) as a function of the ratio between the non-ionic phospholipid 2 and the phospholipid 3. These compounds are illustrated in FIG. 1 and FIG. 14A.

Moreover, the inventors investigated the redox properties of the vesicles using scanning electrochemical microscopy (SECM), dynamic light scattering (DLS) and tunneling electron microscopy (TEM). The inventors also characterized the active redox sites on the vesicle using fluorescent microscopy.

### Instrumentation

Electrochemistry:

Cyclic voltammetry (CV) and scanning electrochemical microscopy (SECM) measurements were performed with an HEKA Electrochemical Probe Scanner 3 or Probe Scanner 1 (HEKA Elektronik Dr. Schulze GmbH, Germany). Pt, Glassy carbon (GC) and Au disk electrodes (CH instruments inc, USA) were used for CV experiments while an homemade 25 μm Pt ultra microelectrode (UME) [62] was used for the SECM experiments. Pt wire and Ag/AgCl wire were used as a counter and reference electrodes, respectively. Prior to experiments, the electrodes were polished using a TegraPol-25 grinder/polisher (Struers Ltd., Mississauga, Canada) equipped with a silicon carbide grinding paper (1200 grit) or alumina slurry (1 and 0.05 μm size).

Spectroscopy:

Dynamic light scattering (DLS) was performed with BI-2005M light scattering system (Brookhaven, UK). Transmission electron microscopy was done with Tecani-TEM (FEI, USA). All vesicles optical microscopy and fluorescence images were done using Zeiss Axio Imager 2 and electrode images were taken using Zeiss Axio Vert. Al (Zeiss, Germany). Fluorescence intensity measurements were conducted using Varian Cary Eclipse Fluorescence spectrometer (Agilent Technologies, USA). Centrifugation was done using IEC CL31 Multispeed Centrifuge (Termo Scientific, USA). Vesicles extrusion was done using Avanti Mini-extruder with a 100 nm pore membrane (Avanti, USA). ¹H, ¹³C and ³¹P NMR spectra were recorded at 300, 75 and 122 MHz, respectively, in CDCl₃ solutions using a Bruker NMR (Bruker, Canada). Mass-spectra measurements were done using 1100 series LC-MSD TOF mass analyzer using an electrospray (ESI) detector (Agilent, USA).

Flow Cytometry:

Measurements were conducted using BD FACSAriallu (BD Biosciences, Canada) and the acquired data was treated with the FlowJo V10 analysis software. The cells were prepared and treated in the same manner as the cells for the fluorescence imaging with one major difference: following exposure to doxorubicin loaded GUVs (each vesicle contained approximately 42.4 μg.mL⁻¹ of doxorubicin) in DMEM−, the solution was removed and the cells were washed with 3 mL of PBS. To harvest the cells, 1 ml of accutase was added to the petri dishes and incubated for an additional 5 minutes followed by an addition of 3 mL of PBS. The cell solution was then transferred into falcon tubes for 5 minutes centrifugation at 1500 rpm. The supernatant was then removed and cells were re-suspended in 1 mL of PBS before the sample was transferred in a polystyrene falcon tube through a 35 μm cell strainer cap. Flow cytometry for doxorubicin (λₑₓ=488 nm, λₑₘ=590 nm) fluorescence detection was done with a blue laser (excitation of 488 nm) and a detector equipped with an emission filter with a bandwidth of 610 nm±10. The samples' fluorescence was compared to auto-fluorescence of untreated cells (not exposed to the non-redox or redox GUVs). All the samples were gated in order to avoid the presence of debris in the final analysis. Flow cytometry results were normalized according to the mode in the FlowJo software. This normalization is accomplished by dividing the histograms of a population in different bins on the x-axis (fluorescence intensity). Each bin of the population is then divided by the bin with the maximum peak of the same population corresponding to the mode (bin containing the highest amount of cell counts). This result is multiplied by 100 to obtain a percentage allowing comparison between samples. Separations: Centrifugation was performed using IEC CL31 Multispeed Centrifuge (Thermo Scientific, USA). Vesicle extrusion was done using Avanti Mini-extruder with a 100 nm pore membrane (Avanti Polar Lipids, Inc., USA).

### Materials

Electrodes:

Pt (Ø=2 mm), Glassy carbon (GC, Ø=3 mm) and Au (Ø=2 mm) disk electrodes (CH instruments Inc., USA) were used for CV experiments, whereas a homemade 25 μm diameter Pt microelectrode (UME) [39] was employed as working electrode for the SECM experiments. A 0.5 mm Pt wire was used as a counter while a 1 mm Ag/AgCl wire (that was made following a method described elsewhere [40]) was used as the reference electrode.

Chemicals:

Compounds 1,2-distearoyl-sn-glycero-3-phosphocholine (DSPC, 2) and 1,2-distearoyl-sn-glycero-3-phospho-(1′-rac-glycerol) (sodium salt) (DSPG, 4) were purchased from Avanti (USA) and Aldrich (Canada). Goat anti-Rabbit IgG, H/L Chains Antibody was obtained from Novus Biologicals (USA). Rabbit anti ferrocene antibody was produced in University of Toronto [27]. 1,2-dimyristoyl-sn-glycero-3-phosphoethanolamine (DMPE), calcein AM, 5(6)-carboxyfluorescein, potassium hexachloroiridate ((III) and (IV)), doxorubicin hydro-chloride (98-100%), tetrabutylammonium hexafluorophosphate (TBA-PF₆), sucrose and D-glucose were purchased from Sigma-Aldrich (Canada). Sodium chloride and potassium chloride were purchased from Fisher Scientific (Canada). Chloroform and anhydrous diethyl ether were purchased from Merck (Canada). All aqueous solutions were prepared from ultrapure filtered water using a Milli-Q Reference purification system (EMD Millipore, USA).

### Example 1— Preparation of Ferrocene Modified Phospholipid (3)

Ferrocene modified phospholipid (FC-DSP) was prepared in the following manner: triethylamine (0.077 mmol, 0.01 mL, 1.4 eq) and N,N-dicyclohexylcarbodiimide (0.077 mmol, 15.9 mg, 1.4 eq) were added to a solution that contained 1,2-dimyristoyl-sn-glycero-3-phosphoethanolamine (0.055 mmol, 35 mg, 1.0 eq) and ferroceneacetic acid (0.077 mmol, 18.8 mg, 1.4 eq) in anhydrous DCM (1.5 mL). The reaction was stirred overnight, until NMR indicated conversion to the coupling was completed. The solution was concentrated under vacuum and then was purified on iatrobeads gel chromatography (10% MeOH: DCM). A dark-brown oil (29.4 mg, 0.0341 mmol) was obtained (62% yield). ¹H NMR (300 MHz, CDCl₃) δ 7.04 (br, 1H), 5.23 (br, 1H), 4.37 (br, 1H), 4.22 (br, 2H), 4.12 (br, 5H), 3.94 (br, 2H), 3.49 (br, 4H), 3.28 (br, 2H), 3.05 (br, 4H), 2.28 (br, 4H), 1.58 (br, 4H), 1.25 (br, 40H), 0.87 (t, J=6.5 Hz, 6H). ³¹P NMR (122 MHz, CDCl₃) δ 0.15 (s). ¹³C NMR (75 MHz, CDCl₃) δ 173.60 (s), 173.21 (s), 70.56 (s), 69.26 (s), 68.92 (s), 68.14 (s), 62.82 (s), 45.87 (s), 34.44 (s), 34.25 (s), 32.05 (s), 29.80 (s), 29.65 (s), 29.49 (s), 29.29 (s), 25.02 (s), 22.81 (s), 14.24 (s), 8.73 (s). HRMS (ESI): Calc. for C₄₅H₇₆FeNO₉P (M+H)⁺: 862.4680; found: 862.4624.

### Example 2—Preparation of Unilamellar Vesicles (Non Redox-Sensitive)

Unilamellar vesicles were prepared as described by Correia-lido and Mauzeroll [28]. More specifically, vial A that contained 3 μmol of DSPC (25.3 mM in CHCl₃), 1 μmoles of DSPG (31.2 mM in CHCl₃) 1 mL of sucrose (215 mM, 240 mOsm) and 1 mL of CHCl₃ and vial B that contained 3 μmol of DSPC (25.3 mM in CHCl₃), 1 μmoles of DSPG (31.2 mM in CHCl₃) 2.5 mL of sucrose (215 mM, 240 mOsm) and 0.5 mL of diethyl ether were vortexed separately (Vial A for 45 seconds and Vial B for 15 seconds) and together for 10 second. The mixture was transferred to a round bottom flask and was heated in a sand bath for 90 minutes at 65-70° C. under a slow Ar flux. The solution was then centrifuged for 30 minutes at 1500 rpm and the vesicles were formed at the solutions interface.

### Example 3—Preparation of Redox-Sensitive Vesicles (5)

Redox vesicles were prepared following the same procedure as described above in Example 2 with an adjustment, namely, each vial contained different ratio of FC-DSP (3) and DSPC (2).

### Example 4—SECM Experiments

SECM feedback mode approach curves were carried out to confirm the electrochemical activity of the giant unilamellar vesicles (GUVs). The experiments were performed in 50 mM glucose solution containing 0.1 M KCl (as electrolyte) and 1 mM Ir^{(III)}Cl₆ (as mediator). The experiments were performed in a glucose solution in order to promote the GUVs immobilization at the bottom of the electrochemical cell during the chronoamperometry experiments. This was possible due to the different in the density between sucrose solution (ρ_{sucrose}=1.59 g.cm⁻³) in the vesicles internal void and glucose (ρ_{glucose}=1.54 g.cm⁻³) in the outer medium. After addition of 50 to 100 μL GUVs solution, the microelectrode was prepositioned using the optical display and a bias potential of 830 mV vs. Ag/AgCl, which is the mediator oxidation potential, was applied to the probe. The approach curve, at a speed of 1 μm s⁻¹, was then acquired above glass in close proximity of the target GUV. The microelectrode was then retracted to a tip to substrate distance of 100 μm. At this distance both the electrode tip as well as target GUVs can be monitored simultaneously using the SECM integrated optical microscope. After positioning the probe above a single GUV of interest, an approach curve was recorded at a scan speed of 1 μm s⁻¹ until a deformation or movement of the GUV was observed due to physical contact with the microelectrode. Five different GUVs were investigated and a comparison of the probe approach behavior with theoretical values in literature systematically confirmed the redox mediator regeneration by the underlying GUV [41].

### Example 5—Indirect Immunofluorescence Imaging

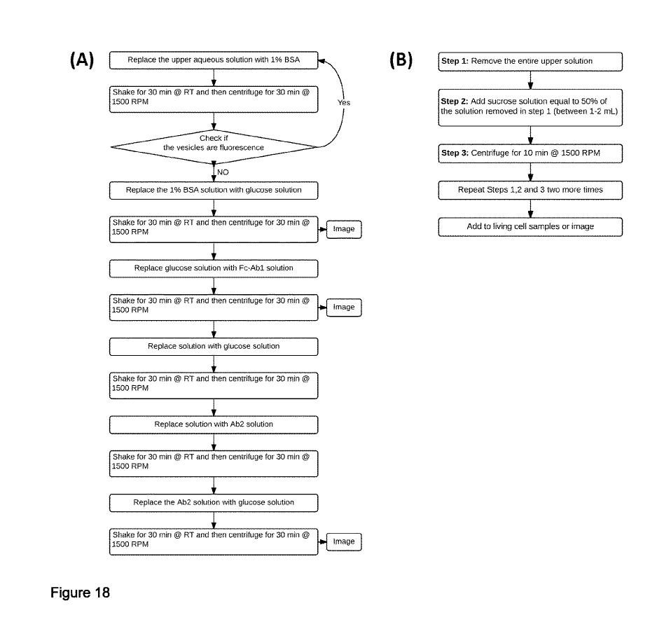

Following the formation of the vesicles (preparation of non-redox (procedure A) and redox (procedure B) vesicles), the upper aqueous phase was replaced using a syringe with a 1% (w/v) bovine serum albumin (BSA) solution in glucose solution (0.1 M KCl and 50 mM glucose) in order to block nonspecific binding. Furthermore, the GUVs demonstrated auto-fluorescent properties that were suppressed due to the BSA binding [42]. The complete mixture was lightly shaken for 30 minutes at RT followed by 30 minutes centrifugation at 1500 rpm. If the vesicles still possessed noticeable fluorescence this procedure was repeated. The aqueous solution of the mixture was then replaced by a BSA-free glucose solution. To remove any BSA leftovers, the complete mixture was centrifuged for 30 minutes at 1500 rpm. The glucose solution is next replaced with a 1% anti-ferrocene rabbit antibody solution in glucose and shaken for 30 minutes at RT followed by 30 minutes centrifugation at 1000 rpm. To remove any residual Fc-Ab1 the aqueous solution was replaced with fresh glucose solution twice. After each re-placement the entire solution was lightly shaken for 15 minutes and then centrifuged at 1000 rpm for 30 minutes. The last step was the introduction of the fluorescence tagged goat anti-rabbit antibody (Ab2). The aqueous solution was replaced with 1:1000 Ab2 in glucose solution, mildly shaken for 30 minutes followed by a 30 minutes centrifugation at 1000 rpm. The top (aqueous) layer was finally replaced with a 50 mM glucose solution (containing 0.1 M KCl) in order to remove any residual antibody and centrifuged for 30 minutes at 1000 rpm. A detailed step diagram is illustrated in FIG. 18A.

### Example 6—Dye Loaded Vesicles

Two different types of dye were used in structural characterization (permeability, stability and triggering) studies.

Calcein:

Due to previous experience with the dye [43,44], calcein AM (55 μM dissolved in a sucrose solution (215 mM, 240 mOsm)), was chosen to monitor vesicle stability and leakage. Before the imaging of the vesicles, the aqueous solution (sucrose) was replaced 2-3 times in order to dilute any free dye in solution and decrease background fluorescence in order to dilute the free dye in the solution and decrease the background noise.

5(6)-carboxyfluorescein (56CF):

This compound was chosen in order to measure the vesicles permeability due to the dye ability to change its fluorescence properties with the pH change [38]. The vesicles were prepared with the dye, using a sucrose solution that was adjusted to pH 10 with NaOH and contained 0.2 mM 56CF. After obtaining the redox and non-redox GUVs which contained the 56CF, the solution was filtered using the micro extruder and a solution of LUV's was obtained.

Doxorubicin Loaded Vesicles:

Both redox and non-redox vesicles were prepared as described above (vesicle preparation, Procedures A and B) with one major difference: 100 and 150 μL of 1 mg.mL⁻¹ solution of doxorubicin, dissolved in sucrose (215 mM, 240 mOsm), was added to vial A and vial B respectively (each of the vials contained a final concentration of 42 μg.mL⁻¹ doxorubicin). In order to reduce the influence of free doxorubicin (both on the cell death rate and on the fluorescence imaging), and to remove the organic phase (chloroform based, which is toxic for the cells), the following steps were taken prior to the addition of the vesicles to the live cells: the aqueous solution was removed and sucrose solution, equal to 50% of the removed volume, was added followed by 10 minutes centrifugation at 1500 rpm. This step was repeated twice until reducing the aqueous solution to approximate 15% of its initial volume. Then the organic phase was removed completely, leaving only the vesicles and the diluted aqueous solution. This is illustrated in FIG. 18B.

### Example 7—Immunoarray Fluorescence Imaging

Following the formation of the vesicles as described above at Examples 2 and 3, aqueous phase was replaced with a 1% (w/v) bovine serum albumin (BSA) solution in glucose solution (0.1 M KCl and 50 mM glucose) in order to quench the vesicle auto-fluorescent and block nonspecific binding. The mixture was lightly shaken for 30 minutes at RT followed by 30 minutes centrifugation at 1500 rpm. If the vesicles still possess high level of fluorescent, this procedure needed to be repeated if the aqueous solution was not replaced by the glucose solution to wash any BSA leftovers and centrifuged for 30 minutes at 1500 rpm.

The next step is the replacement of the glucose solution by the anti-ferrocene rabbit antibody (Fc-Ab1) 1:100 dilution in glucose solution, the mixture was lightly shaken for 30 minutes at room temperature and followed by 30 minutes centrifugation at 1000 rpm. To wash any Fc-Ab1 leftovers, we conducted two washing steps where the aqueous solution was replaced with a glucose solution, lightly shaken for 15 minutes and then centrifuged at 1000 rpm for 30 minutes. The last step was the introduction of the fluorescence tagged goat anti-rabbit antibody (Ab2). The aqueous solution was replaced with 1:1000 Ab2 in glucose solution, lightly shaken for 30 minutes followed by a 30 minutes centrifugation at 1000 rpm. The aqueous solution was replaced one last time with glucose solution and centrifuged for 30 minutes at 1000 rpm in order to wash any antibodies leftovers.

### Example 8—Cell Culture and Fluorescence Imaging

Adenocarcinoma cervical cancer cells HeLa (CCL-2, American Type Culture Collection, VA, USA) were cultured in Dulbecco's Modified Eagle's Medium (DMEM high glucose, Gibco Life Technologies, NY, USA), which contained 10% v/v fetal bovine serum (Sigma-Aldrich, Canada), 2 mM glu-tamine, penicillin and streptomycin (50 units.mL⁻¹) (GE Healthcare Life Sciences' HyClone, UT, USA). Normal lung fibroblast cells (MRC-5, kindly provided by Prof. Sleiman, McGill University, QC, Canada) were cultivated in the same medium without antibiotics to obtain a better growth rate. Cells were grown in tissue culture flasks (Sar-stedt Inc., QC, Canada) and incubated at 37° C. and 5% CO₂, under a water saturated atmosphere. At a confluence of 70% cells were washed once with phosphate buffered saline (PBS, Sigma-Aldrich, pH 7.4) and harvested using 0.25% (v/v) trypsin-ethylenediaminetetraacetic acid (EDTA, Sigma-Aldrich) or accutase (BD Biosciences, Canada). Cells were seeded into 15 mm×60 mm Petri dishes (200,000/dish) and incubated at 37° C. and 5% CO₂ for 18 hours. Prior to the fluorescence imaging, cells were washed once with cell medium lacking serum (DMEM−). Exposed to 20 μL doxorubicin (minimal concentration of 120 μg.mL⁻¹) loaded vesicles (redox and non-redox) in DMEM−, cells were incubated for 5 hours at 37° C. and 5% CO₂. After incubation the solution was removed and substituted for PBS. Fluorescent imaging was conducted using an upright research microscope for advanced imaging Axio Imager 2 (Zeiss, Canada).

### Example 9—Electrode Pretreatment

Pt, Au and GC electrodes were polished with alumina slurry (1 and 0.05 μm) and washed with ultrapure filtered water. Pt and Au electrodes were electrochemically treated in 0.1 M H₂SO₄ by cycling between the oxidation and reduction of water (−0.3 V to 0.9 V for Au, and −0.3 V to 1.1 V for the Pt) until a reproducible voltammetric curve was obtained [45]. Pt microelectrodes were mechanically polished (200 rpm, 4000 grit silicon carbide grinding paper, 15 minutes) until the Pt wire was exposed as a disk. The diameter of the microelectrode was characterized using cyclic voltammetry (3 cycles, −100 mV to +500 mV, 5 mV s⁻¹) in 1 mM K₃Ir^{(III)}Cl₆ (in 0.1 M KCl). The RG of the microelectrode, defined as the ratio of the radius of the entire microelectrode (glass and Pt wire) to that of the Pt metal wire, was determined by optical microscopy.

Referring to FIG. 1, this figure shows the complete formation and triggering of the GUVs. 1,2-Distearoyl-sn-glycero-3-phophocholine (DSPC, 2) is linked to the ferroceneacetic acid (1), by a direct coupling process mediated by N,N′-dicyclohexylcarbodiimide (DCC) in presence of ferroceneacetic acid (1). We concluded that the hydrophilic amino segment of the available phospholipid 2 represented a suitable linker to introduce the redox-active part. A direct coupling process mediated by DCC in presence of 1 produced the desired modified phospholipid 3 in 62% yield. An advantage of this synthesis was the ability to produce the desired target in a single, reproducible and rapid step and in good yield. As will be understood by a skilled person, other synthesis processes may also be performed.

In the subsequent step, phospholipid 3 was mixed together with phospholipid 2 and 1,2-distearoyl-sn-glycero-3-phosphoglycerol (DSPG, 4) in a mixture of solvents and heated for 90 minutes to allow the self-assembly of the redox-active unilamellar vesicles (5).

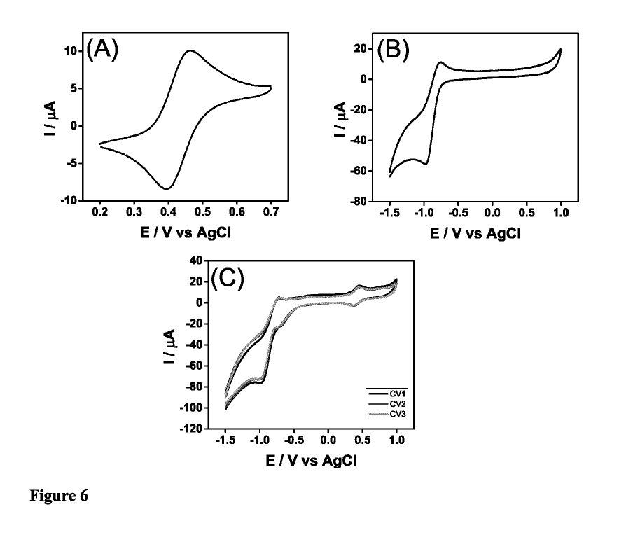

Phospholipid 3 was characterized using both ¹H and ¹³C NMR. Mass spectra and electrochemical measurements were also obtained. FIG. 6 shows the cyclic voltammetry (CV) measurements that were conducted using 1 (FIG. 6A), phospholipid 2 (FIG. 6B) and redox active phospholipid 3 (FIG. 6C) in acetonitrile solution which contained 0.1 M of tetrabutylammonium hexafluorophosphate (TBA-PF₆).

TBA-PF₆ exhibited a reversible oxidation-reduction wave at approximately −1.0 V. When looking at the electrochemical response of phospholipid 2 in presence of TBA-PF₆ (FIG. 6B), the oxidation-reduction wave at approximately −1.0V was dampened due to the presence of the phospholipid in solution. Upon phospholipid modification with ferrocene (Fc), as presented in FIG. 6C, the reversible oxidation reduction of the Fc could be seen at 0.42 V, which was identical to the oxidation reduction of 1 as seen in FIG. 6A. Moreover, at the same time the effect of phospholipid 2 on the oxidation of TBA-PF₆ was also apparent. It was clear from these results that phospholipid 3 was obtained in high purity and exhibited both the properties of phospholipid 2 and 1. The voltammograms were reproducible as illustrated by the three consecutive scans in FIG. 6C. When using Pt or Au electrodes (not shown) similar effects were observed.

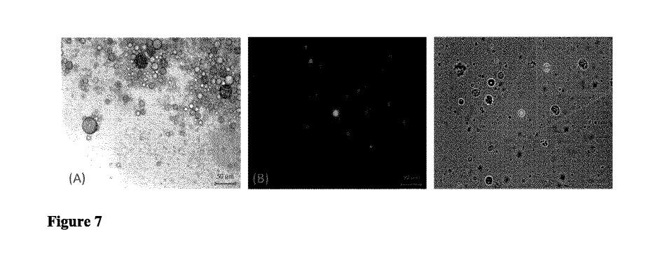

Phospholipid 3 was used to produce the redox-active GUVs. FIG. 7A shows a micrograph of the obtained redox-active GUVs. In order to examine the loading possibilities and the durability of the vesicles according to the invention, we decided to load the GUVs with calcein, a fluorescent dye. The GUVs were produced using the same protocol, with a difference, namely, the sucrose solution contained 55 μM of calcein. The concentration of the dye was lower than what is found in the literature [29,30], since we wanted to avoid an extensive solvent replacement and at the same time to reduce the background noise. Even though we lowered the dye concentration, we still had to perform extractions between 2-3 solvents (only the aqueous part) and to use a more diluted GUVs sample to overcome the background noise. FIG. 7B shows the fluorescent micrograph that was obtained, and FIG. 7C shows an overlay of a fluorescent and transmitted light (TL) micrograph. As can be seen from the images, the GUVs may be used to encapsulate aqueous solutions; accordingly, they may be used as drug delivery vessels.

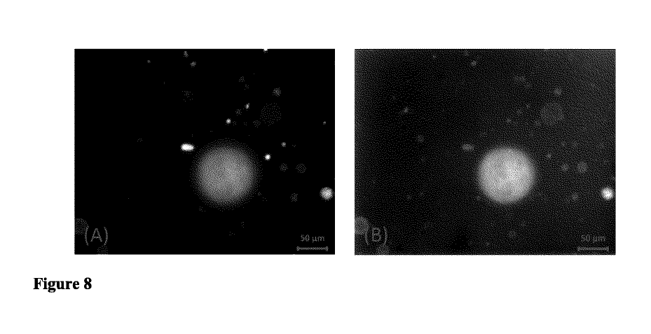

In order to ensure the stability of the vesicles according to the invention to external interferences such as light, we exposed the calcein loaded GUVs (both redox-active and non-active) to excitation by light during different periods (from 1 to 10 minutes). FIG. 8 shows the result of such light-stability experiment. In some cases, photo bleaching was observed but no explicit leakage of the dye was observed; accordingly, the GUVs structure remained intact.

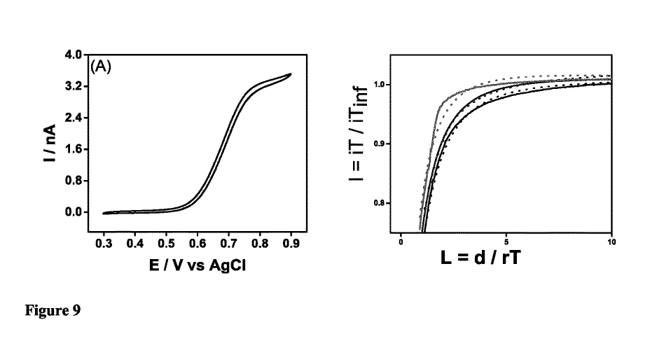

Previous results obtained in our lab showed that the redox-active GUVs exhibited a good electron transfer with the oxidize form of iridium hexachlorate [28]. FIG. 9A shows the CV that was done in the bulk solution of 1 mM K₃Ir^{(II)}Cl₆ in 0.1 M KCl and 50 mM glucose using a 25 μM Pt ultra-microelectrode (Pt-UME). The CV did not reach a stable steady state current in the anodic region due to oxygen generation. Based on the voltammogram in FIG. 9A, a potential of 800 mV was chosen for the SECM approach experiments. FIG. 9B displays SECM approach curves that were done in the same solution as described in FIG. 9A. The black curve represented a negative feedback that was obtained when approaching the glass substrate before adding our GUVs. This curve overlapped the theoretical curve (black-dash line) with apparent heterogeneous electron exchange kinetics (K⁰ₐₚₚ) of 0.0001. We repeated the same experiment this time approaching a single GUV. Two different populations of GUVs were examined, the blue curve represents a GUV with a phospholipid (2):phospholipid (3) ratio of 1:0.2, and the red curve represents a GUV with a phospholipid (2):phospholipid (3) ratio of 1:0.4 (the theoretical curves are also plotted).

The obtained K⁰ₐₚₚ was 0.066 (blue) and 0.105 (red). Both approach curves are still negative despite the fact that the mediator was recycled at the GUV since the effect of the substrate was still dominated leading to the obtained negative feedback curves. Nevertheless the substantial differences in the K were evidence that the GUVs were redox-active and that there were Fc groups on the surface of the GUV (i.e., Fc groups formed an outer layer of the GUV).

Moreover when increasing the amount of phospholipid (3), the K⁰ₐₚₚ increased due to increasing amount of Fc on the surface. However, when we doubled the amount of phospholipid (3) the K⁰ₐₚₚ increased by a factor of 1.5, which suggests that not all the Fc are located on the GUV external surface.

When plotting the theoretical curve of the 1:0.4 ratio GUVs it is clear that these approach curves are not perfectly overlapping. As will be understood by a skilled person, not all of the GUVs are perfectly symmetric, and although the inspected GUVs diameter was smaller than that of the electrode, the geometry of the GUVs will influence the redox recycling. Moreover it is clear by the shape of the curves the ratio (d/a), where d is the distance between the electrodes and a is the electrode active radius, between the curves is fundamentally different.

The GUVs (ratio 1:0.04) were also examined under TEM using an EDX probe in order to provide a further proof that at least part of the ferrocene was exposed on the GUVs surface and not imbedded inside the bilayer or in the internal void. The analysis indeed concluded that the GUVs surface contained iron. The obtained iron weight percent was between 0.13±0.03 to 0.33±0.03. This led us to the conclusion that possibly not all of the iron was exposed to the surface, and some of the Fc groups were indeed inside the vesicle itself, or that the GUVs were not all identical regarding the distribution of phospholipid (3) in the vesicle structure. Also as will be understood by a skilled person, the sizes of the GUVs were not identical, which may affect the number of Fc groups and their distribution on the vesicle surface.

### Example 10

To establish a better understanding of the system according to the invention, we decided to visualize the Fc groups that were exposed on the vesicles surface using an immunoarray fluorescence imaging. We used a polyclonal rabbit anti-ferrocene antibody (Fc-Ab1) to label the ferrocene and a secondary antibody, fluorescent tagged goat anti-rabbit antibody (Ab2), to visualize these specific antibody binding sites. The GUVs themselves possessed a fluorescent ability at the same excitation wave length as Ab2, therefore we used a 1% (w/v) bovine serum albumin (BSA) solution to quench this ability prior to the treatments with the antibodies.

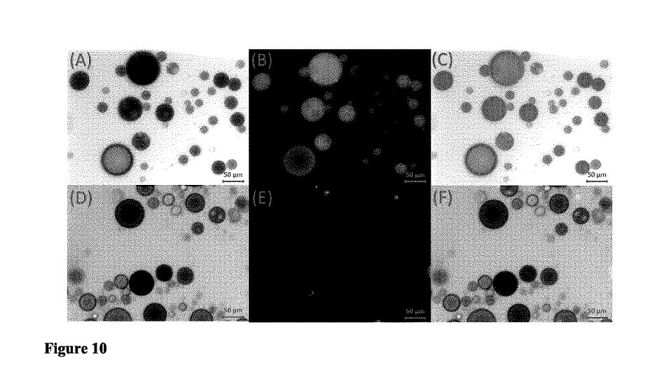

FIG. 10 shows the result of the imaging experiments. FIGS. 10A-C show the result of redox-active GUVs (ratio of 1:0.1 between phospholipid 2 and phospholipid 3), the fluorescent which originated from the Ab2 was demonstrated. At the same time regular GUVs (which were formed in the absence of phospholipid 3) served as a control group remained quenched (FIGS. 10D-F). A review of the prior art [31-34] suggests that it is not trivial for the antibody, due to their size or charge, to cross the membrane by passive transport. These images, together with the fact that the antibodies do not migrate across the membrane, provided another evidence that the ferrocene moiety could be found in the outer hydrophilic shell of the redox active GUVs.

We hypothesized that by an oxidation of the Fc groups that were exposed on the surface of the GUVs we could influence the structural stability [35] of the entire GUVs, resulting in a controlled payload release as suggested in FIG. 1. We prepared both redox-forms of iridium hexachloride and their behaviors were examined. When we titrated the GUVs using the reduced form, K₃Ir^{(III)}Cl₆, as expected, the GUVs were not affected and no payload release was observed. When the oxidized form, K₂Ir^{(IV)}Cl₆, was added to the GUVs solution we observed a payload release response that could be accelerated using larger concentration of K₃Ir^{(III)}Cl₆.

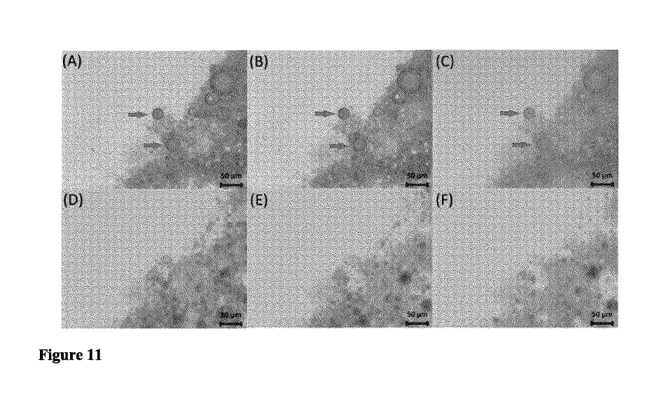

FIG. 11 shows a population of redox-active GUVs before (FIG. 11A) and after (FIGS. 11B-F) the addition of K₃Ir^{(II)}Cl₆. FIGS. 11B-F demonstrates the realization of the hypothesis we suggested, the Fc groups were oxidized and this causes a conformational change in GUVs bilayer resulting in a structural collapse. The response was rapid and in less than 1 second most of the observed population had reacted with the reducing agent. Seven different ratios between phospholipid 2 and phospholipid 3 were used: 1:0.6, 1:04, 1:0.3, 1:0.2, 1:0.1, 1:0.05, 1:0.01. When the ratio was equal to or larger then 1:0.05 good payload release was observed, but at the low ratio of 1:0.01 not all the GUVs demonstrated the payload release, i.e., the concentration of the ferrocene on the GUVs surface was not significant enough to induce a conformational change in the bilayer. One the other hand a high concentration of Fc (for example a ratio of 1:0.6) may lead to more sensitive GUVs that could undergo a fast or unspecific payload release. In an embodiment of the invention, the ratio of 1:0.4 was chosen as the optimal ratio for further experiments.

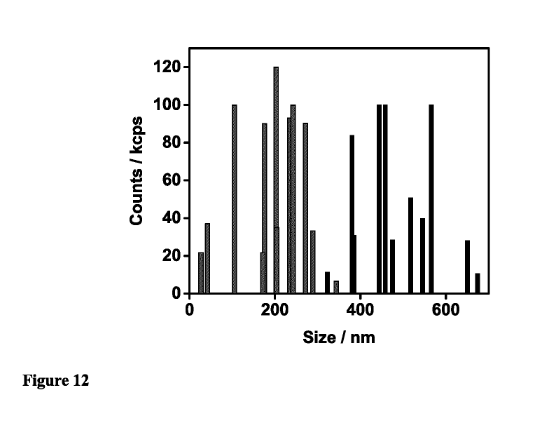

In an embodiment of the invention, the redox-active GUVs may be used for biological systems. To this end, it is desirable that they be of smaller size, in the range of hundreds of nm (i.e., large unilamellar vesicles (LUV) or small unilamellar vesicles (SUV)) [36,37]. We used the Avanti microextroder to filter our solutions. The filtered samples were examined using dynamic light scattering (DLS). FIG. 12 shows the result of a DLS experiments of using a 1:0.4 ratio redox active LUV before and after adding 45 μM of K₃Ir^{(IV)}Cl₆. A dramatic change in the average size, from 450-500 nm to 200 nm, was noted. As will be understood by a skilled person, the change was attributed to the oxidation reaction the LUV followed by a structural change as explained above. When K₃Ir^{(III)}Cl₆ was added, no significant size change was observed.

### Example 11

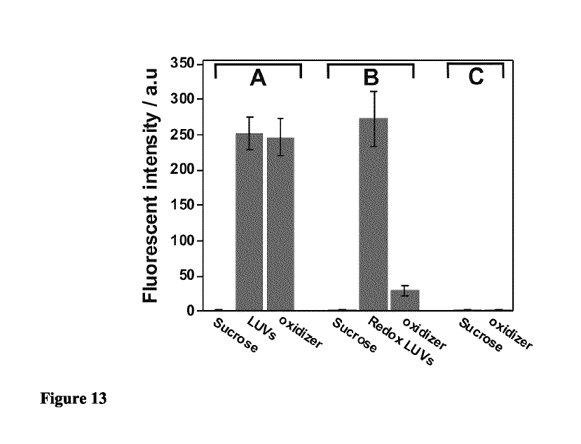

In order to further demonstrate the payload release of the redox vesicles according to the invention, we used 5-6 carboxyfluorescein (56CF), a pH sensitive dye [38]. We prepared two different types of vesicles, regular (non redox-active) and redox-active, both were preloaded with the 56CF dye. The vesicles that contained an inner solution of sucrose that contained 0.2 mM of 56CF in pH 10 were filtered using the microextruder and a solution of LUVs was obtained. For each sample we preformed three types of measurements: background which consisted from 2 mL of sucrose that was adjusted to pH 4 using HCl, vesicles by adding 100 μL of the LUVs to the sucrose solution, and oxidizer by adding 150 μL of 1 mM Ir^{(IV)}Cl₆²⁻ (dissolved in sucrose pH 4) and we measured the fluorescent intensity. FIG. 13 shows the results of these measurements.

When we added regular LUVs (group A) there was an increase in the fluorescent intensity due to the presence of the encapsulated dye. When adding our oxidizer, Ir^{(IV)}Cl₆²⁻, no significant change in the intensity was observed since the oxidizer does not interact with the vesicles. When we repeated this experiment with the redox-active LUVs (group B), again we saw an increase in the intensity when adding the vesicles to the sucrose. But unlike the regular LUVs, the addition of the oxidizer caused the release of the dye to the outer solution. The change in the pH environment affected the dye and a change in the fluorescent intensity was absorbed. Indeed, we observed a 90% decrease in the fluorescent. As will be understood by a skilled person, a reason the fluorescent intensity is still higher than the background solution may be attributed to dye that is imbedded in the bilayer and not exposed to the outer solution or vesicles that did not contain redox-active groups on their outer shell. Another type of experiment we preformed was adding the oxidizer to the sucrose solution (group C), and as expected this did not influence the fluorescent intensity.

Further embodiments of the invention are described below.

### Redox Active Phospholipid Synthesis and Characterization

Formation of the redox active phospholipid is illustrated in FIG. 14A. 1,2-dimyristoyl-sn-glycero-3-phosphoethanolamine (DSPE, 2) is linked to ferroceneacetic acid (1), using a direct coupling process mediated by N,N′-dicyclohexylcarbodiimide (DCC). The hydrophilic amino segment of 2 was deemed a good linker to introduce the redox active moiety and resulted in the generation of a polar amide functionality that had similar hydrophilic properties to that of a natural phospholipid. The direct coupling process mediated by DCC in presence of 1 produced the desired modified phospholipid 3 in 62% yield in a single reproducible and rapid step.

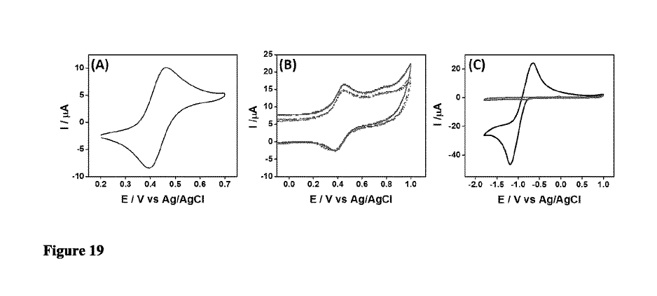

Phospholipid 3 was characterized using NMR, mass spectrometry and electrochemistry as outlined above (FIG. 14B). The CV of 1 (FIG. 19A), phospholipid 2 and redox active phospholipid 3 (FIG. 14B) were performed in acetonitrile containing 0.1 M of tetrabutylammonium hexafluorophosphate (TBA-PF₆) used as an electrolyte. In the TBA-PF₆ electrolyte, a clear reversible oxidation-reduction wave at −0.9 V (FIG. 19C) was observed which was attributed to dissolved oxygen reduction in nonaqueous solution mediated by the TBA⁺ ions [46] and was damped after purging the solution for 30 minutes using N₂. Following the coupling of the phospholipid with the ferrocene moiety (FIG. 14A), the reversible electrochemistry of ferrocene is observed at 0.42 V (FIG. 14B), which is consistent with the electrochemistry observed for 1 (FIG. 19A). Given its stable and reversible electrochemical behavior (FIG. 19B), the use of phospholipid 3 as a redox trigger was further investigated in self-assembled vesicles, so-called redox bearing vesicles.

### Formation and Triggering of Redox-Bearing Vesicles

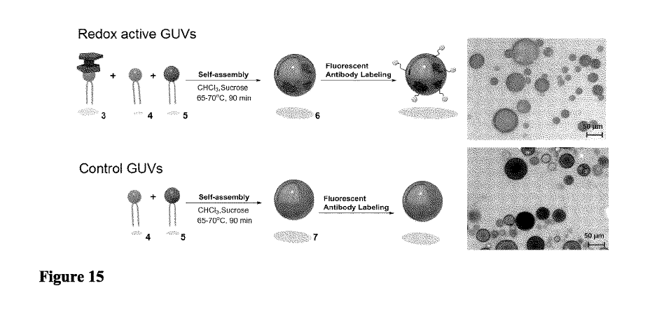

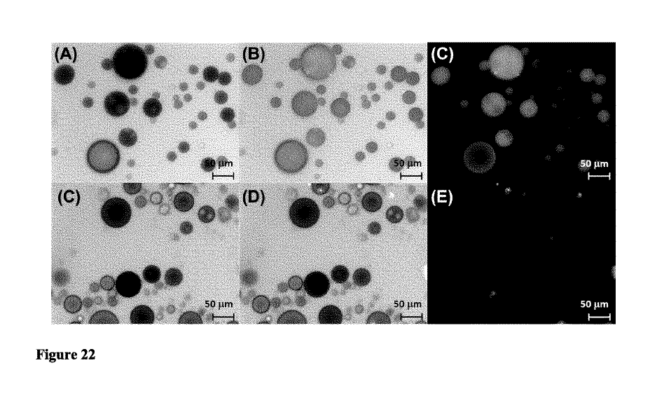

Formation of the redox bearing vesicles 6 (FIG. 15) was achieved through the double emulsion method [47] by mixing phospholipid 3 with 1,2-distearoyl-sn-glycero-3-phosphocholine (DSPC, 4) and 1,2-distearoyl-sn-glycero-3-phosphoglycerol (DSPG, 5) in a heated mixture of solvents as described in the experimental section (vesicle preparation). Initially, the formation, characterization and triggering studies of the self-assembled vesicles were carried out on giant unilamellar vesicles (GUVs), which have amenable dimensions for optical and fluorescent microscopy. Optical micrographs (FIG. 15 and FIG. 22) confirmed the spherical and polydisperse (100 nm-40 μm) nature of the prepared GUVs.

The effective loading, impermeability to large molecules and overall stability of the GUVs were confirmed from fluorescent microscopy imaging using calcein AM (λₑₓ=496 nm, λₑₘ=516 nm) [48,43] encapsulated GUVs. Calcein AM was pre-loaded in the internal void of GUVs during self-assembly. The fluorescent micrograph, which was acquired on a low density GUV sample to limit spectral interferences, confirms the successful encapsulation of calcein AM. The GUVs stability and impermeability to large molecules was confirmed by fluorescent time lapse imaging of GUV populations for 1-hour periods that did not result in increased fluorescence in the outer GUV solution, which may be caused by vesicle leakage. In calcein AM photo-bleaching [49] controls monitored in both redox and non-redox active GUVs fluorescence decrease in the internal void of the GUV was observed without explicit calcein AM leakage.

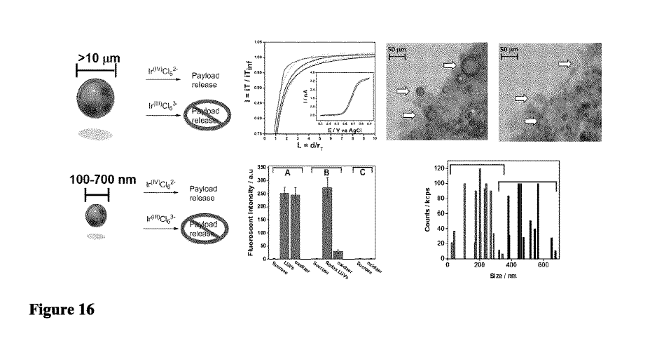

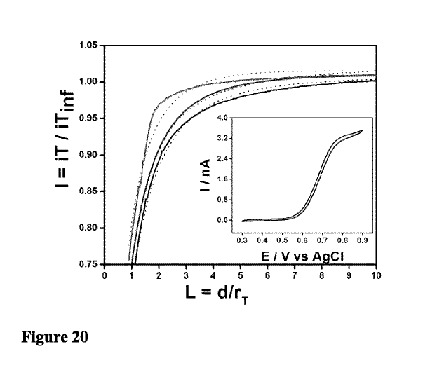

To investigate the redox triggering properties of vesicles 6, the ferrocene moieties of the GUVs, which are exposed to the outer solution, were oxidized through a controlled electrochemical titration that locally produced the oxidizing agent, K₃Ir^{(IV)}Cl₆ [28]. Specifically, SECM approach curve measurements were acquired in feedback mode. From the CV preformed in 1 mM K₃Ir^{(III)}Cl₆ (FIG. 16 and FIG. 20), an oxidizing potential of 830 mV was applied at the 25 μm Pt working microelectrode. The applied potential far exceeded the standard electrode potential of the K₃Ir^{(III)}Cl₆/K₂Ir^{(IV)}Cl₆ couple (+0.6 V vs. Ag/AgCl/0.1M KCl [50]), effectively imposing diffusion limited conditions. As a control, an approach curve was recorded above pure glass (FIG. 16 and FIG. 20, black curves) and experimental data (full line) was compared to theoretical negative feedback approach in literature (dotted line) [51]. The obtained experimental control displays a negative feedback consistent with existing SECM feedback theory [52].

The integrated optical microscope of the SECM was used to position the working electrode above single GUVs. In FIG. 16 and FIG. 20, a representative single GUV measurement taken from two different populations were studied: a GUV, prepared with a phospholipid 4:phospholipid 3 ratio of 1:0.2 (full blue curve) and a GUV, prepared with a ratio of 1:0.4 (full red curve). An enhanced regeneration of the K₃Ir^{(III)}Cl₆ is observed for both types of GUVs, since the recorded approach curves are well above the pure negative feedback behavior. As expected, the ferrocene moieties of the GUVs that are exposed to the external solution are able to reduce the K₂Ir^{(IV)}Cl₆ supplied by the microelectrode. Apparent heterogeneous kinetic (k⁰ₐₚₚ) values were extracted (where the diffusion coefficient of the iridium complex was 7.5.10⁻⁶ cm² s⁻¹ [53]) for both redox active GUVs, whereby phospholipid ratios of 1:0.2 and 1:0.4 result in k⁰ₐₚₚ values of 2.0.10⁻⁴ cm.s⁻¹ (blue) and 3.2.10⁻⁴ cm.s⁻¹ (red), respectively. The theoretical approach curves for corresponding first order heterogeneous kinetics (dotted lines) present small deviations from experimental curves because of experimental uncertainties related to GUV curvature, tip to GUV distance or nonhomogeneous surface distribution of ferrocenes.

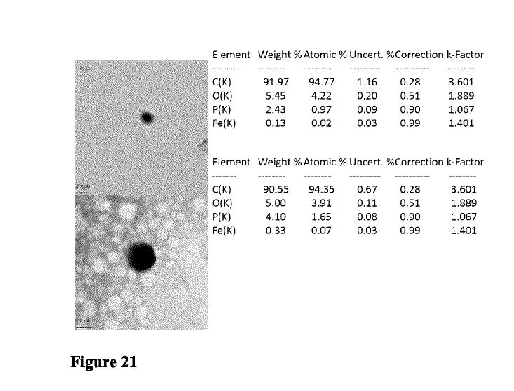

To corroborate the SECM findings confirming that the ferrocene moieties of the GUVs were facing the external solution, GUVs (ratio 1:0.04) were examined under TEM using an EDX probe. Based on the measurements of 8 different vesicles, the EDX analysis confirmed that the surface of the GUVs contained an iron weight percentage ranging from 0.13 to 0.33±0.03%, (FIG. 21). A range of ferrocene surface density is expected given the broad size distribution of the GUVs. A fraction of the ferrocene moieties may be present in the inner void of the GUVs (either during the formation or due to a conformational change). Following a short incubation in BSA to prevent nonspecific binding [54,42], immunofluorescence microscopy of the GUVs was performed. A polyclonal rabbit anti-ferrocene antibody (Fc-Ab1) was used to label the external ferrocene [27] and a secondary antibody, fluorophore-tagged (λₑₓ=493 nm, λₑₘ=518 nm) goat anti-rabbit antibody (Ab2), was used to visualize these specific antibody binding sites (FIG. 15). The immunofluorescence labels external ferrocenes since it is unlikely that the antibody would cross the GUV membrane by passive transport given its size and charge [33,34]. When treating redox-active GUVs (ratio of 1:0.1 between phospholipid 4 and phospholipid 3) with the Fc-Ab1 and Ab2, the presence of ferrocene moieties was demonstrated, as presented in FIGS. 22A-C. Non-redox active GUVs (7), which were formed in the absence of phospholipid 3, and served as a control group remained non-fluorescent (FIGS. 22D-F). FIG. 21 and FIGS. 22A-F, combined with the unlikely presence of antibody in the GUVs internal void confirms the presence of the ferrocene moiety in the outer hydrophilic shell of the redox active GUVs.

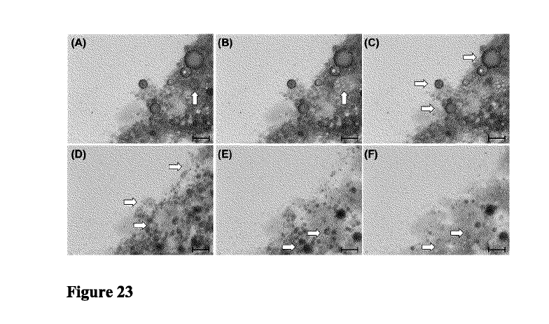

The triggering mechanism of the redox GUVs is based on the increase coulombic repulsion interactions between the positively charged ferrocenium, which destabilize the structural stability [35] of the GUVs, resulting in a controlled payload release (FIG. 16). When the GUVs (redox or non-redox active) were titrated using the reduced form, K₃Ir^{(II)}Cl₆, no payload release was observed. When the oxidized form, K₂Ir^{(IV)}Cl₆, was added to the GUVs (redox or non-redox active), a fast evident payload release response was observed (FIG. 16 and FIG. 23) only with the redox active GUVs. Prior to the addition of K₂Ir^{(IV)}Cl₆, a population of redox-active GUVs were marked for comparison in FIG. 23A. Upon addition of K₂Ir^{(IV)}Cl₆ (FIGS. 23B-F), a time dependent payload release of GUVs is observed. FIG. 16 and FIGS. 23A-F clearly support the claim that the ferrocene groups were oxidized and this causes a conformational change in the GUVs bilayer resulting in a structural collapse. The payload release response occurred in less than 1 second whereby most of the observed GUVs population had reacted with K₂Ir^{(IV)}Cl₆. Seven different ratios between phospholipid 4 and phospholipid 3 were investigated: 1:0.6, 1:04, 1:0.3, 1:0.2, 1:0.1, 1:0.05, 1:0.01. When the ratio was equal to or larger than 1:0.05, efficient payload release was observed. At the lower ratio than 1:0.01 partial payload release was observed as a result of low surface density of ferrocenes that could not induce a significant conformational change in the bilayer. A ratio of 1:0.4 was chosen as the optimal ratio for future experiments.

### Adjusting Vesicle Size for Drug Delivery Applications

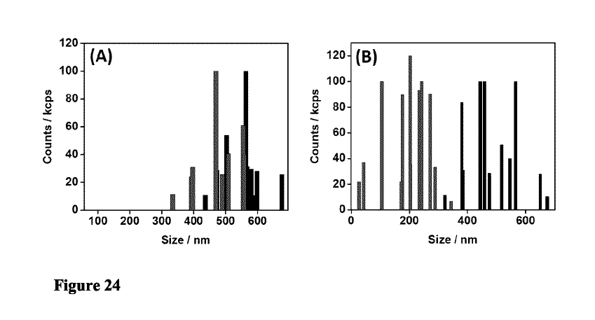

While GUVs are a good model system for in vitro observation, their size is a limiting factor for in vivo application [36,37]. There is evidence that large unilamellar vesicles (LUVs) are more efficient then small unilamellar vesicles (SUVs) since the curvature and dimensions of the SUVs increases the surface tension and reduces their stability [55]. Furthermore, there is a well-defined size range requirement (200-1200 nm [56]) in order to extravasate through leaky blood vessels in tumors by enhanced permeability and retention (EPR). This structural abnormalities near the tumor vasculature combined with poor or a lack of lymphatic drainage (EPR effect) [57,58] increases the efficiency and selectivity of the LUVs towards the tumor site. After preparing the LUVs, the samples were examined using DLS. The result of the DLS experiments (FIG. 16 and FIG. 24) using a 1:0.4 ratio of redox active LUVs before and after adding 45 μM of K₂Ir^{(IV)}Cl₆ confirmed that the filtered assembly were also able to release their cargo based on the dramatic change in the measured average size from 500 nm to 200 nm. These results were consistent with what was observed in the GUVs, the oxidation reaction of the ferrocene moieties at the surface of the LUVs is responsible for the payload release. The addition of a reduced complex (K₃Ir^{(III)}Cl₆) to a new population of redox LUVs did not affect the payload release and no size change was observed.

In order to further demonstrate the payload release of the redox vesicles 5(6)-carboxyfluorescein (56CF), a pH sensitive dye [38], was used. Non-redox and redox active LUVs containing 56CF were prepared. Both samples were added to a 2 ml sucrose solution at pH 4 and the fluorescence intensity was measured before (as a control) and after the addition of the LUVs. The fluorescent intensity was also measured after an addition of an oxidizer (150 μL of 1 mM K₂Ir^{(IV)}Cl₆ dissolved in sucrose pH 4), seen in FIG. 16. When the non-redox active LUVs (group A) were added to the sucrose solution an increase in the fluorescence intensity is observed due to the presence of the encapsulated dye. Upon addition of K₂Ir^{(IV)}Cl₆, no significant change in the intensity was observed, since they do not contain any ferrocene moieties and the oxidizer does not interact with the vesicles. When repeating the same experiment with the redox active LUVs (group B), a substantial raise in the intensity when adding the vesicles to the sucrose is also detected. Upon addition of K₂Ir^{(IV)}Cl₆ a 90% decrease in fluorescence is observed showing the efficiency of the redox selective payload release mechanism. The observed residual fluorescence intensity is attributed to small fraction of dye imbedded in the bilayer and not exposed to the extra-vesicular solution.

### In Vitro Assay of Doxorubicin Loaded Redox GUVs in HeLa Cells

Tumors are associated with heterogeneous vascularization (expressed as the EPR effect), leading to hypoxia. The reduced oxygen concentration in the cancer cell environment interferes with redox-related reactions, for example incomplete oxygen reduction, which is reflected by a decreased mitochondrial transmembrane potential [59,60]. Furthermore, an increase in the reactive oxygen species (ROS) production may be observed thus effecting the local redox environment [61].

To demonstrate that the presence of cancer cells could trigger the payload release of the redox vesicles due to the local redox gradient, both types of GUVs (redox and non-redox active) were pre-loaded with doxorubicin, a common chemotherapy drug with a narrow therapeutic index, that has serious cardiotoxic side effects [48].

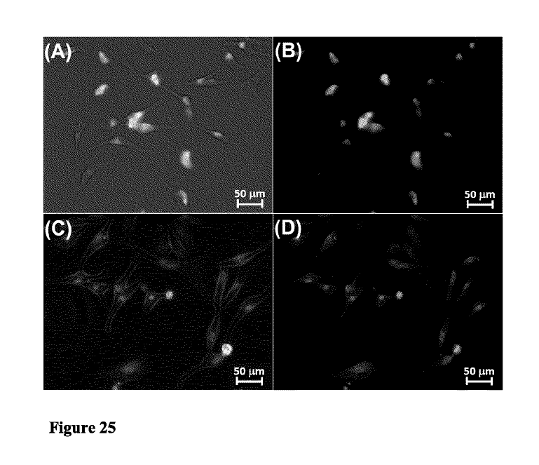

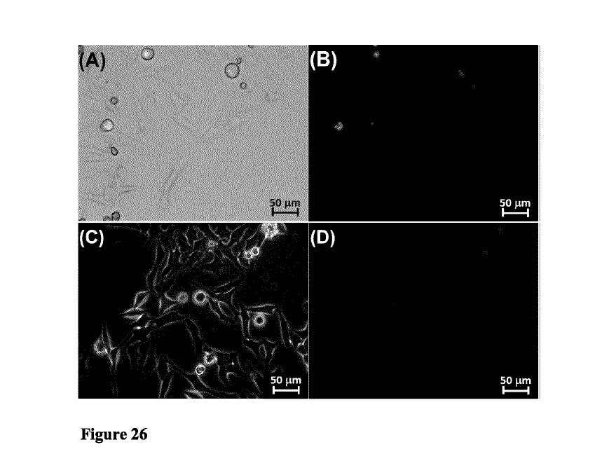

After a 5-hour incubation period, the cells were washed and imaged. FIG. 25 shows the bright field and dark field optical micrographs of HeLa cells treated with redox active GUVs. Doxorubicin has a strong fluorescent signal at 590 nm [43], which was monitored in order to evaluate the cells' drug uptake and the efficiency of the GUVs payload release. In all cases, there was a vast uptake of the doxorubicin by the cells, confirming that the altered redox state of cancer cells [44] elicits a payload release. In the control experiments involving non-redox GUVs (FIG. 26), negligible doxorubicin uptake by the cells was observed.

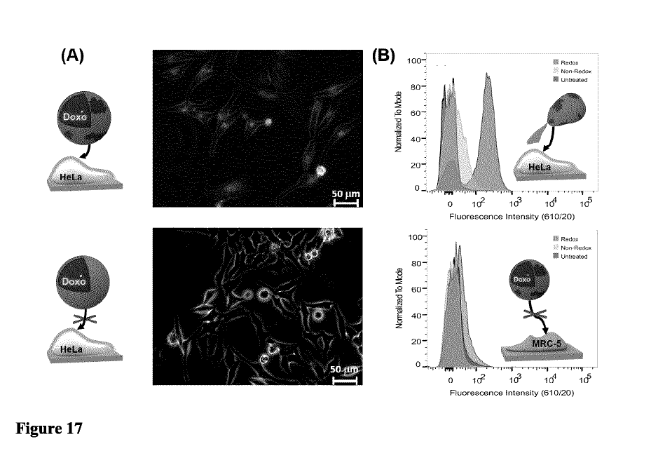

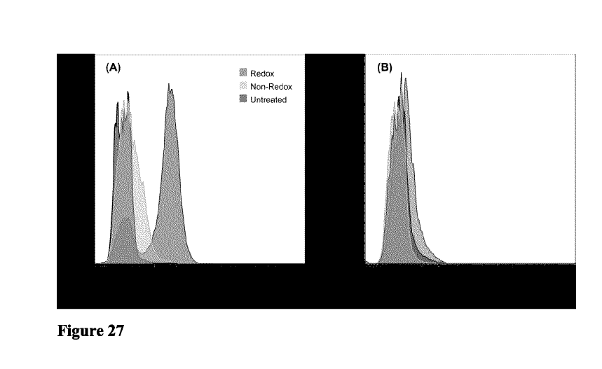

To establish a statistically valid comparison between the redox-active GUVs (FIG. 25) and the non-redox GUVs (FIG. 26), the fluorescence intensity of both populations compared to untreated HeLa cells was measured by flow cytometry (FIG. 17). Additionally, to establish the selectivity of the redox active GUVs to cancer cells, a flow cytometry experiment comparing cancerous HeLa and non-cancerous MRC-5 cells exposed to redox and non-redox GUVs or untreated (control population) was performed (FIG. 17 and FIG. 27). As shown on FIG. 17 and FIG. 27A, the fluorescence intensity of the HeLa cells treated with redox active GUVs is 200 times higher than the one of the untreated cells. The redox histogram presents 2 different peaks because the number of cells analyzed purposely far exceeded the number of GUVs added to the sample. The first peak is assigned to cells showing a fluorescence intensity similar to the untreated cells whilst the second peak shows the cells that included the doxorubicin released by the GUVs. When comparing the median of the peak corresponding to the redox GUV exposition, one can see the signal is 10 times stronger than the one of the cells treated with the non-redox GUVs. We assume that even in the non-redox GUVs one may expect unspecific payload release due to changes in the osmolality between the solutions or due to different physical interferences (mixing, pipetting, temperature changes). This might explain the difference between the non-redox GUVs and the untreated cells where the non-redox GUVs show a fluorescence intensity of about 15 times more intense than the untreated cells. As for MRC-5 non-cancerous cells (FIG. 17 and FIG. 27B), all the samples show low median of fluorescence intensity where the redox-exposed cells are twice as much fluorescent than the untreated cells which is a hundred times lower than seen in the HeLa cells. As for the non-redox sample its fluorescence median is too close to the one of the untreated to be distinguished and when compared to the redox sample it is only 3 times smaller. An important finding is that the cancer cells (HeLa) treated with redox GUVs show a signal with a full order of magnitude higher than the one of the non-cancerous cells (MRC-5). These results (FIG. 17 and FIGS. 25-27) show that the payload mechanism is specific to cancer cells and when coupled with the EPR effect it potentially leads to a new technique of dealing with cancer-related diseases.

As will be understood by a skilled person, the present disclosure provides for the preparation of a ferrocene modified phospholipid, in a single step reaction. The phospholipid was purified and characterized using spectroscopic as well as electrochemical methods. This phospholipid was later used to produce a redox active unilamellar vesicle. These structures according to the invention were characterized using advanced methods such as SECM, TEM and immunofluorescence imaging. It was also proven that the ferrocene groups were exposed on the surface of the vesicle and thus accessible for redox triggering. The GUVs also showed stability when reduced to LUVs (matching the recommended dimension for cancer targeted vesicles). Using DLS and fluorescent measurements the payload release was observed and it was shown that these vesicles (LUVs) still performed well even in smaller scales making them promising candidates for a redox-triggered drug delivery system. When pre-loading the GUVs with an anti-cancer agent (doxorubicin) and exposing them to live cancerous HeLa and non-cancerous MRC-5 cells, the selectivity of the payload mechanism towards cancer cells was demonstrated.

Although the present invention has been described hereinabove by way of specific embodiments thereof, it can be modified, without departing from the spirit and nature of the subject invention as defined in the appended claims.

The present description refers to a number of documents, the content of which is herein incorporated by reference in their entirety.

### REFERENCES

1. Abidian, M. R., D. H. Kim, and D. C. Martin; Conducting-polymer nanotubes for controlled drug release; Advanced Materials, 2006 18(4), p. 405-+.
2. Fry, N. L., G. R. Boss, and M. J. Sailor; Oxidation-Induced Trapping of Drugs in Porous Silicon Microparticles; Chemistry of Materials 2014 26(8), p. 2758-2764.
3. Song, J., et al.; Redox responsive nanotubes from organometallic polymers by template assisted layer by layer fabrication; Nanoscale 2013 5(23), p. 11692-11698.
4. Svirskis, D., et al.; Electrochemically controlled drug delivery based on intrinsically conducting polymers; Journal of Controlled Release 2010 146(1), p. 6-15.
5. Medina, O. P., Y. Zhu, and K. Kairemo; Targeted liposomal drug delivery in cancer; Current Pharmaceutical Design 2004 10(24), p. 2981-2989.
6. Allen, T. M. and P. R. Cullis; Liposomal drug delivery systems: From concept to clinical applications; Advanced Drug Delivery Reviews 2013 65(1), p. 36-48.
7. Pinzon-Daza, M. L., et al.; Nanoparticle- and Liposome-carried Drugs: New Strategies for Active Targeting and Drug Delivery Across Blood-brain Barrier; Current Drug Metabolism 2013 14(6), p. 625-640.
8. Offerman, S. C., et al.; Ability of co-administered peptide liposome nanoparticles to exploit tumour acidity for drug delivery; Rsc Advances 2014 4(21), p. 10779-10790.
9. Maurer-Spurej, E., et al.; Factors influencing uptake and retention of amino-containing drugs in large unilamellar vesicles exhibiting transmembrane pH gradients; Biochimica Et Biophysica Acta-Biomembranes 1999 1416(1-2), p. 1-10.
10. Castelli, F., et al.; A calorimetric study on diflunisal release from poly(lactide-co-glycolide) microspheres by monitoring the drug effect on dipalmitoylphosphatidylcholine liposomes: Temperature and drug leading influence; Drug Delivery 2000 7(1), p. 45-53.
11. Leserman, L. D., et al.; Targeting to cells of fluorescent liposomes covalently coupled with monoclonal antibody or protein-A; Nature 1980 288(5791), p. 602-604.
12. Martin, F. J., W. L. Hubbell, and D. Papahadjopoulos; Immunospecific targeting of liposomes to cells—A novel and efficient method for covalent attachment of fab′ fragments via disulphide bonds; Biochemistry 1981 20(14), p. 4229-4238.
13. Davidsen, J., et al.; Secreted phospholipase A(2) as a new enzymatic trigger mechanism for localised liposomal drug release and absorption in diseased tissue; Biochimica Et Biophysica Acta-Biomembranes 2003 1609(1), p. 95-101.
14. Harnoy, A. J., et al.; Enzyme-Responsive Amphiphilic PEG-Dendron Hybrids and Their Assembly into Smart Micellar Nanocarriers; Journal of the American Chemical Society 2014 136(21), p. 7531-7534.
15. Noble, G. T., et al.; Ligand-targeted liposome design: challenges and fundamental considerations; Trends in Biotechnology 2014. 32(1), p. 32-45.
16. Klibanov, A. L., et al.; Ultrasound-triggered release of materials entrapped in microbubble-liposome constructs: A tool for targeted drug delivery; Journal of Controlled Release 2010 148(1), p. 13-17.
17. Paasonen, L., et al.; Gold-embedded photosensitive liposomes for drug delivery: Triggering mechanism and intracellular release; Journal of Controlled Release 2010 147(1), p. 136-143.
18. Thompson, D. H., et al.; Triggerable plasmalogen liposomes: Improvement of system efficiency; Biochimica Et Biophysica Acta-Biomembranes 1996 1279(1), p. 25-34.
19. a) U.S. 2011-0104250, b) EP 1225873, c) U.S. Pat. No. 6,726,925, d) U.S. 2011-0275980, e) U.S. 2012-0129270
20. Saji, T., K. Hoshino, and S. Aoyagui; Reversible formation and disruption of micelles by control of the redox state of the head group; Journal of the American Chemical Society 1985 107(24), p. 6865-6868.
21. Rosseto, R. and J. Hajdu; A rapid and efficient method for migration-free acylation of lysophospholipids: synthesis of phosphatidylcholines with sn-2-chain-terminal reporter groups; Tetrahedron Letters 2005 46(16), p. 2941-2944.
22. Rosseto, R., et al.; Synthesis of phosphatidylcholine analogues derived from glyceric acid: a new class of biologically active phospholipid compounds; Tetrahedron Letters 2008 49(21), p. 3500-3503.
23. Loew, M., J. C. Forsythe, and R. L. McCarley; Lipid Nature and Their Influence on Opening of Redox-Active Liposomes; Langmuir 2013 29(22), p. 6615-6623.
24. McCarley, R. L.; Redox-Responsive Delivery Systems. Annual Review of Analytical; Chemistry 2012 5, p. 391-411.
25. Ong, W., et al.; Redox-Triggered Contents Release from Liposomes; Journal of the American Chemical Society 2008 130(44), p. 14739-14744.
26. Clegg, A. D., et al.; Marcus theory of outer-sphere heterogeneous electron transfer reactions: High precision steady-state measurements of the standard electrochemical rate constant for ferrocene derivatives in alkyl cyanide solvents; Journal of Electroanalytical Chemistry 2005 580(1), p. 78-86.
27. Martic, S., et al.; Versatile Strategy for Biochemical, Electrochemical and Immunoarray Detection of Protein Phosphorylations; Journal of the American Chemical Society 2012 134(41), p. 17036-17045.
28. Correia-Ledo, D., A. A. Arnold, and J. Mauzeroll; Synthesis of Redox Active Ferrocene-Modified Phospholipids by Transphosphatidylation Reaction and Chronoamperometry Study of the Corresponding Redox Sensitive Liposome; Journal of the American Chemical Society 2010 132(43), p. 15120-15123.
29. Tamba, Y. and M. Yamazaki; Single giant unilamellar vesicle method reveals effect of antimicrobial peptide magainin 2 on membrane permeability; Biochemistry 2005 44(48), p. 15823-15833.
30. Tamba, Y., et al.; Single GUV method reveals interaction of tea catechin (−)-epigallocatechin gallate with lipid membranes; Biophysical Journal 2007 92(9), p. 3178-3194.
31. Weill, C. O., S. Biri, and P. Erbacher; Cationic lipid-mediated intracellular delivery of antibodies into live cells; Biotechniques 2008 44(7), p. Pvii-Pxi.
32. Dalkara, D., G. Zuber, and J. P. Behr; Intracytoplasmic delivery of anionic proteins; Molecular Therapy 2004 9(6), p. 964-969.
33. Weill, C. O., et al.; A practical approach for intracellular protein delivery; Cytotechnology 2008 56(1), p. 41-48.
34. Zelphati, O., et al.; Intracellular delivery of proteins with a new lipid-mediated delivery system; Journal of Biological Chemistry 2001 276(37), p. 35103-35110.
35. Jin, Z. Y., et al.; Electrochemically Controlled Drug-Mimicking Protein Release from Iron-Alginate Thin-Films Associated with an Electrode; Acs Applied Materials & Interfaces; 2012 4(1), p. 466-475.
36. Rhoden, V. and S. M. Goldin; Formation of unilamellar lipid vesicles of controllable dimensions by detergent dialysis; Biochemistry 1979 18(19), p. 4173-4176.
37. Rodrigueza, W. V., et al.; Large versus small unilamellar vesicles mediate reverse cholesterol transport in vivo into two distinct hepatic metabolic pools—Implications for the treatment of atherosclerosis; Arteriosclerosis Thrombosis and Vascular Biology 1997 17(10), p. 2132-2139.
38. Mordon, S., et al.; Characterization of tumorous and normal tissue using a pH-sensitive fluorescence indicator (5,6-carboxyfluorescein) in vivo; Journal of Photochemistry and Photobiology B-Biology; 1992 13(3-4), p. 307-314.
39. Danis, L., Polcari, D., Kwan, A., Gateman, S. M. & Mauzeroll, J. Fabrication of Carbon, Gold, Platinum, Silver, and Mercury Ultramicroelectrodes with Controlled Geometry. Analytical Chemistry, doi:10.1021/ac503767n (2015).
40. East, G. A. & del Valle, M. A. Easy-to-make Ag/AgCl reference electrode. Journal of Chemical Education 77, 97-97 (2000).
41. S. Kuss, R. Cornut, I. Beaulieu, M. A. Mezour, B. Annabi and J. Mauzeroll, Bioelectrochemistry (Amsterdam, Netherlands), 2011, 82, 29-37.
42. L. Tan, L. Liu, Q. Xie, Y. Zhang and S. Yao, Analytical sciences: The International Journal of the Japan Society for Analytical Chemistry, 2004, 20, 441-444.
43. J. Vaage, D. Donovan, P. Uster and P. Working, Br. J. Cancer, 1997, 75, 482-486.
44. A. Acharya, I. Das, D. Chandhok and T. Saha, Oxidative Med. Cell. Longev., 2010, 3, 23-34.
45. Noyhouzer, T., Valdinger, I. & Mandler, D. Enhanced Potentiometry by Metallic Nanoparticles. Analytical Chemistry 85, 8347-8353, doi:10.1021/ac401744w (2013).
46. C. O. Laoire, S. Mukerjee, K. M. Abraham, E. J. Plichta and M. A. Hendrickson, The Journal of Physical Chemistry C, 2010, 114, 9178-9186.
47. Z. H. Zawada, Cell. Mol. Biol. Lett., 2004, 9, 589-602.
48. C. H. Kim, N. Kim, H. Joo, J. B. Youm, W. S. Park, D. Van Cuong, Y. S. Park, E. Kim, C. K. Min and J. Han, J. Cardiovasc. Pharmacol., 2005, 46, 200-210.
49. D. Correia-Ledo, A. A. Arnold and J. Mauzeroll, Journal of the American Chemical Society, 2010, 132, 15120-15123.
50. N. Song and D. M. Stanbury, Inorganic Chemistry, 2008, 47, 11458-11460.
51. R. Cornut and C. Lefrou, Journal of Electroanalytical Chemistry, 2007, 608, 59-66.
52. A. J. Bard and M. V. Mirkin, Scanning Electrochemical Microscopy, Second Edition, Taylor & Francis, 2012.
53. J. V. Macpherson, C. E. Jones and P. R. Unwin, Journal of Physical Chemistry B, 1998, 102, 9891-9897.
54. Q. Yue, T. Shen, C. Wang, C. Gao and J. Liu, International Journal of Spectroscopy, 2012, 2012, 9.
55. M. Traikia, D. E. Warschawski, O. Lambert, J. L. Rigaud and P. F. Devaux, Biophys. J., 2002, 83, 1443-1454.
56. C. M. Dawidczyk, C. Kim, J. H. Park, L. M. Russell, K. H. Lee, M. G. Pomper and P. C. Searson, Journal of Controlled Release, 2014, 187, 133-144.
57. H. Maeda, H. Nakamura and J. Fang, Advanced Drug Delivery Reviews, 2013, 65, 71-79.
58. H. Maeda, J. Wu, T. Sawa, Y. Matsumura and K. Hori, Journal of Controlled Release, 2000, 65, 271-284.
59. A. M. Abrantes, M. E. S. Serra, A. C. Gonçalves, J. Rio, B. Oliveiros, M. Laranjo, A. M. Rocha-Gonsalves, A. B. Sarmento-Ribeiro and M. F. Botelho, Nuclear Medicine and Biology, 2010, 37, 125-132.
60. M. Weinmann, V. Jendrossek, D. Guner, B. Goecke and C. Belka, FASEB journal: official publication of the Federation of American Societies for Experimental Biology, 2004, 18, 1906-1908.
61. J. M. Lluis, F. Buricchi, P. Chiarugi, A. Morales and J. C. Fernandez-Checa, Cancer research, 2007, 67, 7368-7377.
62. Danis, L.; Polcari, D.; Kwan, A., Gateman, S. M Mauzeroll, J., Analytical Chemistry, 2015, 87(5), 2565-2569.

1. A redox-sensitive compound comprising a redox-sensitive organometallic moiety and a phospholipid or modified-phospholipid moiety, optionally the two moieties are attached together by a linker which is a C₁ to C₈ alkyl group optionally comprising at least one of C═O, C═S, C═N and O═S═O, optionally the backbone of the linker comprises at least one heteroatom selected from O, S and N.

2. A redox-sensitive compound according to claim 1 having a general formula 3A
Q-L-U 3A
wherein:
Q is a redox-sensitive organometallic group, preferably the metal is selected from Fe, Ir, Ru and Pt;
L, which is present or absent, is a linker which is a C₁ to C₈ alkyl group optionally comprising at least one of C═O, C═S, C═N and O═S═O, optionally the backbone of the linker comprises at least one heteroatom selected from O, S and N; and
U is a phospholipid or modified-phospholipid moiety.

3. A redox-sensitive compound according to claim 1 having a general formula 3B, 3C, 3D, 3E or 3F 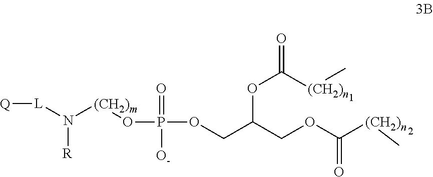
wherein:
Q is a redox-sensitive group comprising at least one metal atom;
L, which is present or absent, is a linker which is a C₁ to C₈ alkyl group optionally comprising at least one of C═O, C═S, C═N and O═S═O, optionally the backbone of the linker comprises at least one heteroatom selected from O, S and N;
R is a C₁ to C₈ alkyl group;
m is an integer selected from 1 to 8; and
n₁ and n₂ are each independently an integer selected from 1 to 30, 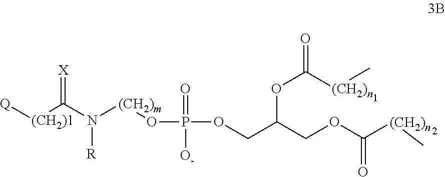
wherein:
Q is a redox-sensitive group comprising at least one metal atom;
X is a heteroatom selected from O, S and N;
R is a C₁ to C₈ alkyl group;
l and m are each independently an integer selected from 1 to 8; and
n₁ and n₂ are each independently an integer selected from 1 to 30, 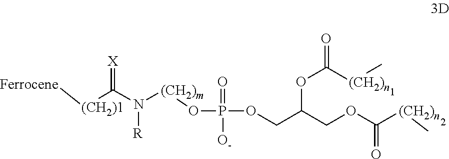
wherein:
X is a hetero atom selected from O, S and N;
R is a C₁ to C₈ alkyl group;
l and m are each independently an integer selected from 1 to 8; and
n₁ and n₂ are each independently an integer selected from 1 to 30, 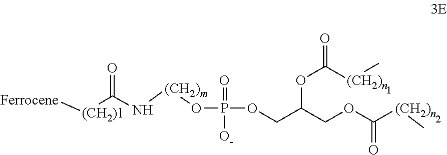
wherein:
l and m are each independently an integer selected from 1 to 8; and
n₁ and n₂ are each independently an integer selected from 1 to 30, 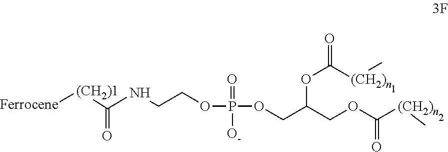
wherein:
l is an integer selected from 1 to 8; and
n₁ and n₂ are each independently an integer selected from 1 to 30.

4.-7. (canceled)

8. A redox-sensitive phospholipid according to claim 1, which is of formula 3

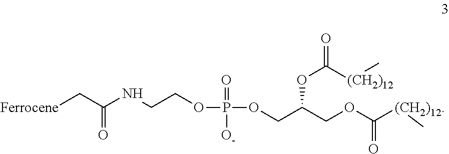

9. A redox-sensitive drug delivery system comprising:
a redox-sensitive compound as defined in claim 1; or
a redox-sensitive compound as defined in claim 1, and at least one phospholipid compound that is not redox-sensitive; or
a redox-sensitive compound as defined in claim 1, and two phospholipid compounds that are not redox-sensitive.

10.-11. (canceled)

12. A redox-sensitive drug delivery system comprising,
a redox-sensitive phospholipid of formula 3E or 3F as defined in claim 3; or
a redox-sensitive phospholipid of formula 3E or 3F as defined in claim 3, and at least one phospholipid compound that is not redox-sensitive; or
a redox-sensitive phospholipid of formula 3E or 3F as defined in claim 3, and first and second phospholipid compounds that are not redox-sensitive.

13.-14. (canceled)

15. A redox-sensitive drug delivery system according to claim 12, wherein, when the redox-sensitive phospholipid is of formula 3E, the first phospholipid compound that is not redox-sensitive is a compound of general formula 2A outlined below and the second phospholipid that is not redox-sensitive is a compound of general formula 4A outlined below; and when the redox-sensitive phospholipid is of formula 3F, the first phospholipid compound that is not redox-sensitive is a compound of general formula 2A′ outlined below 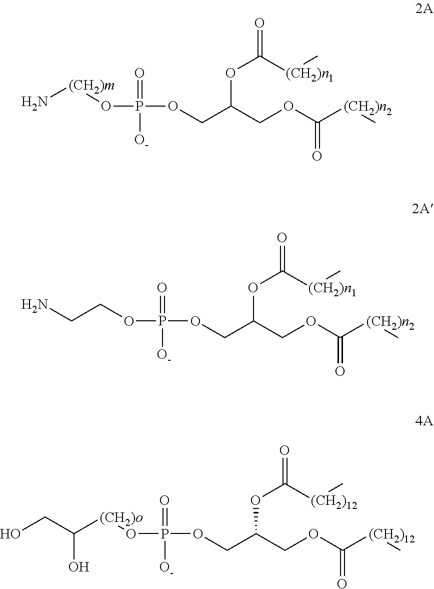
wherein:
m and o and each independently an integer selected from 1 to 8; and
n₁ and n₂ are each independently an integer selected from 1 to 30.

16. A redox-sensitive drug delivery system comprising:
the redox-sensitive phospholipid of formula 3 as defined in claim 8; or
the redox-sensitive phospholipid of formula 3 as defined in claim 8, and at least one phospholipid that is not redox-sensitive; or
the redox-sensitive phospholipid of formula 3 as defined in claim 8, and first and second phospholipid compounds that are not redox-sensitive.

17.-18. (canceled)

19. A redox-sensitive drug delivery system according to claim 16, wherein the first phospholipid compound that is not redox-sensitive is a compound of general formula 2 outlined below and the second phospholipid that is not redox-sensitive is a compound of general formula 4 outlined below

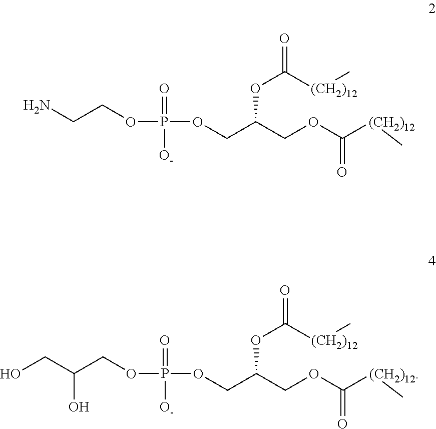

20. A redox-sensitive drug delivery system according to claim 9, wherein at least part of the redox-sensitive groups of the redox-sensitive phospholipid is located on an outer surface of the system.

21. A redox-sensitive drug delivery system according to claim 12, wherein at least part of the ferrocene groups of the redox-sensitive phospholipid 3E or 3F is located on an outer surface of the system.

22. A redox-sensitive drug delivery system according to claim 16, wherein at least part of the ferrocene groups of the redox-sensitive phospholipid 3 is located on an outer surface of the system.

23. A redox-sensitive drug delivery system according to claim 9, wherein:
the redox-sensitive phospholipid and one of the two phospholipid compounds are present in a molar ratio phospholipid compound:redox-sensitive phospholipid between about 1:0.01 to about 1:1; and/or
the system has a size between about 100 nm to 40 μm.

24.-26. (canceled)

27. A method for preparing a redox-sensitive phospholipid of formula 3E, as defined in claim 3 comprising reacting a redox-sensitive compound of formula 1A and a phospholipid of formula 2A as outlined below 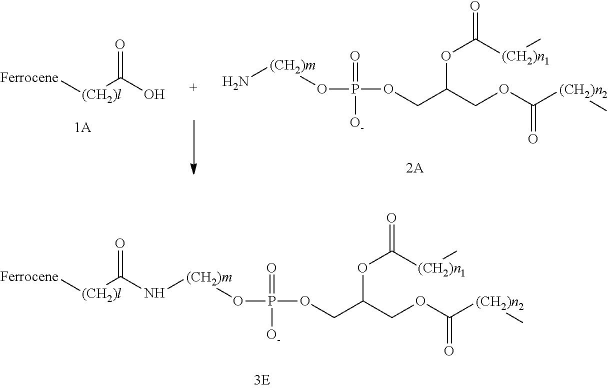
wherein:
l and m and each independently an integer selected from 1 to 8; and
n₁ and n₂ are each independently an integer selected from 1 to 30.

28. A method for preparing a redox-sensitive drug delivery system, comprising (a) providing a redox-sensitive phospholipid of formula 3E as defined in claim 3; and (b) mixing the redox-sensitive phospholipid of formula 3E and a first phospholipid of formula 2A in the presence of a second phospholipid of formula 4A outlined below 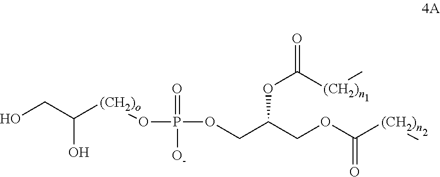
wherein:
o is an integer selected from 1 to 8; and
n₁ and n₂ are each independently an integer selected from 1 to 30.

29.-30. (canceled)

31. A method for preparing a redox-sensitive phospholipid of formula 3, as defined in claim 8 comprising reacting a redox-sensitive compound of formula 1 and a phospholipid of formula 2 as outlined below

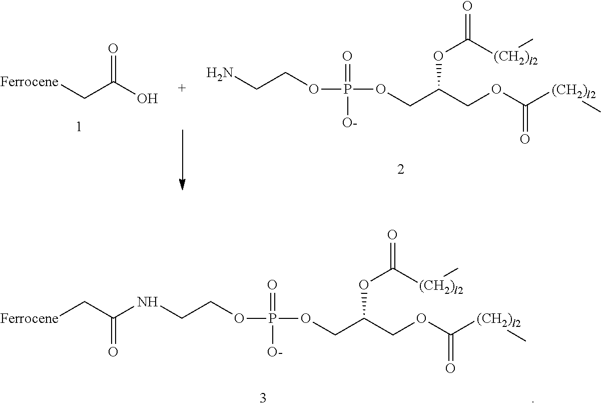

32. A method for preparing a redox-sensitive drug delivery system, comprising: (a) providing a redox-sensitive phospholipid of formula 3 as defined in claim 8; and (b) mixing the redox-sensitive phospholipid of formula 3 and a first phospholipid of formula 2 in the presence of a second phospholipid of formula 4 outlined below

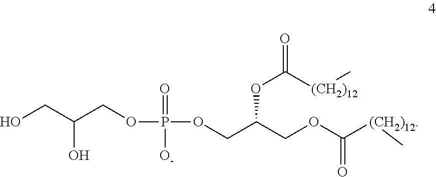

33.-40. (canceled)

41. A redox-sensitive drug delivery system which is obtained by a method as defined in claim 27.

42. A method for preparing a loaded redox-sensitive drug delivery system, comprising: (a) providing a redox-sensitive drug delivery system as defined in claim 9; and (b) mixing the redox-sensitive drug delivery system and a biologically active agent.

43. A method for preparing a loaded redox-sensitive drug delivery system, comprising: (a) providing a redox-sensitive phospholipid of formula 3E as defined in claim 3; and (b) mixing the redox-sensitive phospholipid of formula 3E, a first phospholipid of formula 2A, a second phospholipid of formula 4A, and a biologically active agent.

44. (canceled)

45. A method according to claim 42, wherein:
the biologically active agent is encapsulated within the system; and/or
the biologically active agent is selected from: antitumor agents, antibiotics, anthracycline antibiotics, immunodilators, anti-inflammatory drugs, drugs acting on the central nervous system, proteins, peptides, doxorubicin, daunorubicin, epirubicin, idarubicin, and mitoxantrone.

46.-48. (canceled)

49. A loaded redox-sensitive drug delivery system which is obtained by the method as defined in claim 42.

50. (canceled)

51. A pharmaceutical composition comprising a loaded redox-sensitive drug delivery system as defined in claim 49, and a pharmaceutically acceptable carrier.

52. A method of treating a medical condition in a human or animal, comprising administering to the human or animal a loaded redox-sensitive drug delivery system as defined in claim 49, and wherein the loaded biologically active agent is for treating the medical condition.

53.-55. (canceled)

56. A method according to claim 52, wherein:
the biologically active agent is released upon contact of the loaded system with an oxidant; preferably the oxidant is Ir^{(IV)}Cl₆²; and/or
the biologically active agent is released upon contact of the loaded system with a biological system; and/or
the biologically active agent is released upon contact of the loaded system with cancer cells and the biologically active agent is not released upon contact of the loaded system with other cells.

57.-58. (canceled)

59. A research platform, which embodies:
a redox-sensitive compound as defined in claim 1; or
a redox-sensitive compound as defined in claim 1 and at least one phospholipid compound that is not redox-sensitive.

60.-63. (canceled)

64. A research platform, which embodies a redox-sensitive drug delivery system as defined in claim 9.

65. (canceled)
